(19) United States
(12) Patent Application Publication
(10) Pub. No.: US 2021/0113519 A1
(43) Pub. Date: Apr. 22, 2021

(54) Compositions and Methods for Modulating Permeability of Biological Barriers

(71) Applicant: Carnegie Mellon University, Pittsburgh, PA (US)

(72) Inventors: Kathryn A. Whitehead, Pittsburgh, PA (US); Nicholas G. Lamson, LaFayette, NY (US)

(21) Appl. No.: 17/047,772

(22) PCT Filed: Apr. 17, 2019
(86) PCT No.: PCT/US2019/027866

§ 371 Date: Oct. 15, 2020

Related U.S. Application Data
(60) Provisional application No. 62/762,039, filed on Apr. 17, 2018.

(51) Int. Cl. A61K 31/353; A61K 38/28; A61K 9/00; A61K 36/73

(52) U.S. Cl. CPC A61K 31/353; A61K 36/73; A61K 9/0053; A61K 38/28

(57) ABSTRACT

A method of reversibly increasing intestinal epithelium permeability is provided. A plant polyphenol composition, such as a pelargonidin-containing composition is enterically-administered, e.g., with a high molecular weight therapeutic agent, such as insulin, such that therapeutic agents having high molecular weights can be delivered enterically. An oral dosage form for enteric delivery of high molecular weight therapeutic agents also is provided.

### CROSS REFERENCE TO RELATED APPLICATIONS

This application claims the benefit of U.S. Provisional Patent Application No. 62/762,039, filed Apr. 17, 2018, which is incorporated herein by reference in its entirety.

Provided herein are methods of enhancing enteric drug delivery. Also provided herein are dosage forms providing enhanced enteric drug delivery.

Significant focus is on non-injectable methods to deliver therapeutic macromolecules (e.g. proteins and nucleic acids). One of the major challenges in accomplishing this, however, is increasing the permeability of the relevant biological barriers (e.g. cell membranes, the intestinal epithelium or the skin) that normally prevent the transport of macromolecules. Specifically, although oral delivery is one of the most patient-friendly routes of drug administration, it currently cannot be used for large or highly charged drugs because they do not permeate the intestinal epithelium. One approach to address the issue of limited permeability and uptake is the use of chemical permeation enhancers, which include compounds that promote transport across a biological mass transfer barrier. Previous studies have shown that certain synthetic chemicals can greatly increase the permeability of epithelial membranes, allowing absorption of macromolecule drugs. This increase in permeability can happen either via the transcellular route (through cell membranes and the cell layer) or the paracellular route (in between cells). Paracellular permeability can be enhanced through modulation of the tight junctions, which are dynamic protein structures that connect epithelial cells. Unfortunately, nearly all previously reported permeation enhancers, which include detergents, acids, salts, and nitrogenous small molecules, are associated with toxic side effects to intestinal cells and tissues. This issue has prevented most permeation enhancers from being implemented in clinical delivery applications. Thus, there is a need for compounds and compositions that increase intestinal epithelial permeability, especially for delivery of macromolecular active agents.

### SUMMARY

According to one aspect of the invention, an oral dosage form is provided. The oral dosage form comprises: a therapeutic agent ranging in size from 10 Da to 150,000 Da (150 kDa, or kiloDaltons), from 10 Da to 120 kDa, such as, from 100 Da to 110 kDa, from 100 Da to 80 kDa, from 100 Da to 40 kDa, from 100 Da to 25 kDa, or from 100 Da to 10 kDa, from 1,000 Da to 100,000 Da, from 1,500 Da to 80,000 Da, or from 2,000 Da to 50,000 Da; a polyphenol-containing composition, comprising a polyphenol or a mixture of plant polyphenols able to increase permeability of the intestinal epithelium in a patient, and in an amount effective to increase permeability of intestinal epithelium of a patient; and an enteric coating or delayed-release coating surrounding the therapeutic agent and the polyphenol-containing composition.

In another aspect, a method of delivering an active agent to a patient is provided. The method comprises: enterically delivering to a patient an amount of a polyphenol-containing composition comprising a polyphenol or a mixture of plant polyphenols able to increase permeability of the intestinal epithelium in a patient, in an amount effective to increase permeability of intestinal epithelium of a patient; and delivering a therapeutic agent ranging in size from 10 Da to 150 kDa, from 10 Da to 120 kDa, such as, from 100 Da to 110 kDa, from 100 Da to 80 kDa, from 100 Da to 40 kDa, from 100 Da to 25 kDa, or from 100 Da to 10 kDa, from 1,000 Da to 100,000 Da, from 1,500 Da to 80,000 Da, or from 2,000 Da to 50,000 Da to the intestine at the same time as the delivery of the polyphenol-containing composition to the patient, within 6, 5, 4, 3, 2, or 1 hour, or 30 minutes of delivery of the polyphenol-containing composition to the patient, or during a time period where the permeability of the intestinal epithelium of the patient is increased due to the presence of the delivered polyphenol-containing composition.

In another aspect, a method of reversibly increasing intestinal epithelium permeability in a patient is provided. The method comprises delivering to an intestine of a patient an amount of a polyphenol-containing composition comprising a polyphenol or a mixture of plant polyphenols able to increase permeability of the intestinal epithelium in a patient, in an amount effective to increase permeability of intestinal epithelium of a patient, wherein the polyphenol-containing composition comprises: isolated pelargonidin; a pelargonidin-enriched fraction obtained from a natural plant material; a mixture of isolated or enriched polyphenols comprising pelargonidin; isolated petunidin; a petunidin-enriched fraction obtained from a natural plant material; a mixture of isolated or enriched polyphenols comprising petunidin; isolated malvidin; a malvidin-enriched fraction obtained from a natural plant material; a mixture of isolated or enriched polyphenols comprising malvidin; or any combination thereof.

In yet another aspect, a method of treating hyperglycemia or diabetes in a patient, comprising administering an amount of a dosage form to the patient effective to treat hyperglycemia or diabetes in a patient. The dosage form comprises: insulin; a polyphenol-containing composition, comprising a polyphenol or a mixture of plant polyphenols able to increase permeability of the intestinal epithelium in a patient, and in an amount effective to increase permeability of intestinal epithelium of a patient; and an enteric coating or delayed-release coating surrounding the therapeutic agent and the polyphenol-containing composition.

The following numbered clauses illustrate various aspects of the invention:

Clause 1. A dosage form comprising:
a therapeutic agent ranging in size from 10 Da to 150,000 Da (150 kDa, or kiloDaltons), from 10 Da to 120 kDa, such as, from 100 Da to 110 kDa, from 100 Da to 80 kDa, from 100 Da to 40 kDa, from 100 Da to 25 kDa, or from 100 Da to 10 kDa, from 1,000 Da to 100,000 Da, from 1,500 Da to 80,000 Da, or from 2,000 Da to 50,000 Da; and
a polyphenol-containing composition, comprising a polyphenol or a mixture of plant polyphenols able to increase permeability of the intestinal epithelium in a patient, and in an amount effective to increase permeability of intestinal epithelium of a patient.
Clause 2. The dosage form of clause 1, wherein the polyphenol-containing composition is prepared from a red-pigmented plant material, a pelargonidin-containing plant material, a petunidin-containing plant material, or a malvidin-containing plant material.
Clause 3. The dosage form of clause 1, wherein the red-pigmented plant material is strawberry fruit (the fruit of the strawberry plant, e.g., red strawberries, not an unripe, green strawberry), red tomato, red potato, red grape skin, or a geranium flower.
Clause 4. The dosage form of any one of clauses 1-3, wherein the polyphenol-containing composition is liquidized or crushed fruit, fruit skin, or flower.
Clause 5. The dosage form of any one of clauses 1-3, wherein the polyphenol-containing composition is dried (e.g., lyophilized) and optionally comminuted (e.g. powdered, chopped, macerated, etc.) fruit, fruit skin, or flower.
Clause 6. The dosage form of clause 1, wherein the polyphenol-containing composition is a polyphenol extract of a fruit, a fruit skin, or a flower.
Clause 7. The dosage form of any one of clauses 1-6, wherein the polyphenol-containing composition comprises one or more flavonoids or anthocyanidins or anthocyanins.
Clause 8. The dosage form of clause 7, wherein the polyphenol-containing composition is an anthocyanidin-enriched or anthocyanin-enriched fraction prepared from a fruit, a fruit skin, or a flower.
Clause 9. The dosage form of clause 1, wherein the polyphenol-containing composition comprises pelargonidin or a glycoside thereof, such as callistephin.
Clause 10. The dosage form of clause 1, wherein the polyphenol-containing composition comprises petunidin or a glycoside thereof, such as petunidin-3-O-glucoside.
Clause 11. The dosage form of clause 1, wherein the polyphenol-containing composition comprises malvidin or a glycoside thereof, such as oenin.
Clause 12. The dosage form of clause 1, wherein the polyphenol-containing composition is a fraction prepared from a fruit, fruit skin, or flower that is retained on a hydrophobic resin in water and is eluted from the resin in ethanol.
Clause 13. The dosage form of clause 12, wherein the hydrophobic resin is a macroreticular aliphatic crosslinked polymer composition, such as an acrylic polymer composition.
Clause 14. The dosage form of any one of clauses 1-13, wherein the therapeutic agent is insulin.
Clause 15. The dosage form of clause 14, wherein the insulin is provided in an amount ranging from 5 IU insulin to 200 IU insulin per unit dose.
Clause 16. The dosage form of any one of clauses 1-13, wherein the therapeutic agent is a protein.
Clause 17. The dosage form of any one of clauses 1-13, wherein the therapeutic agent is one or more of abaloparatide, adrenocorticotropic hormone, afamelanotide, albiglutide, ambamustine, atosiban, aviptadil, buserelin, carbetocin, carfilzomib, carperitide, cetrorelix, cholecystokinin, calcitonin (salmon or human), carperitide, corticotropin, cyclosporine, degarelix, desmopressin, dulaglutide, elcatonin, eledoisin, enalapril, enfuvirtide, etelcalcetide, exenatide, felypressin, ganirelix, glatiramer, glucagon, glucagon-like peptide 2, glucose-dependent insulinotropic peptide, gonadorelin, goserelin, heparin, histrelin, human growth hormone, icatibant, lanreotide, leuprorelin (leuprolide), linaclotide, liraglutide, lisinopril, lixisenatide, lucinactant, lutetium, lypressin, mifamurtide, nafarelin, nesiritide, octreotide, ornipressin, oxytocin, pasireotide, plecanatide, pramlintide, romiplostim, romurtide, somatostatin, taltirelin, teduglutide, teriparatide, terlipressin, tetracosactide, thymopentin, triptorelin, vasopression, virus capsid proteins and other antigens, voclosporin, or ziconotide. Specific active ingredients in the binding agents class deliverable by the methods, dosage forms, or compositions described herein include, without limitation: abciximab, adalimumab, alefacept, alemtuzumab, alirocumab, altumomab pentetate, arcitumomab, atezolizumab, avelumab, basiliximab, bectumomab, belimumab, benralizumab, bermekimab, besilesomab, bevacizumab, bezlotoxumab, biciromab, blinatumomab, blontuvetmab, brentuximab vedotin, brodalumab, burosumab, canakinumab, caplacizumab, capromab pendetide, removab, cemiplimab, certolizumab pegol, cetuximab, clivatuzumab tetraxetan, cosfroviximab, daclizumab, daratumumab, denosumab, dinutuximab, dupilumab, durvalumab, eculizumab, edrecolomab, efalizumab, elotuzumab, emapalumab, emicizumab, erunumab, ertumaxomab, etanercept, etracizumab, evolocumab, famolesomab, fantolizumab, fremanezumab, galcanezumab, gemtuzumab ozogamicin, girentuximab, golimumab, guselkumab, ibalizumab, ibritumomab tiuxetan, idarucizamab, igovomab, imciromab, ipilimumab, itolizumab, ixekizumab, labetuzumab, lanadelumab, loviketmab, mepolizumab, mogamulizumab, motavizumab, muromonab-CD3, naptumomab estefenatox, natalizumab, necitumumab, nimotuzumab, nivolumab, nofetumomab, oblitoxaximab, obinutuzumab, ocrelizumab, ofatumumab, olaratumab, omalizumab, oportuzumab, oregovomab, palivizumab, panitumumab, pembrolizumab, pemtumomab, pertuzumab, racotumomab, ramucirumab, ranibizumab, ravulizumab, reslizumab, risankizumab, rituximab, romosozumab, rovelizumab, ruplizumab, sarilumab, secukinumab, siltuximab, sulesomab, tamtuvetmab, tefibazumab, tildrakizumab, toclizumab, tositumomab, trastuzumab, ustekinumab, vedolizumab, visilizumab, votumumab, zalutumumab, or zanolimumab.
Clause 18. The dosage form of any one of clauses 1-13, wherein the therapeutic agent is a binding reagent, such as an antibody fragment.
Clause 19. The dosage form of clause 17, wherein the therapeutic agent is an F(ab) fragment, an F(ab′)₂ fragment, an F(ab′) fragment, an scFv (single-chain variable fragment), a di-scFv (dimeric single-chain variable fragment), a bi-specific T-cell engager (BiTE), a single-domain antibody (sdAb), or an antibody binding domain.
Clause 20. The dosage form of any one of clauses 1-13, wherein the therapeutic agent is a carbohydrate such as a polysaccharide or a glycosaminoglycan.
Clause 21. The dosage form of any one of clauses 1-13, wherein the therapeutic agent is heparin.
Clause 22. The dosage form of any one of clauses 1-21, in the form of an oral dosage form comprising an enteric coating or delayed-release coating surrounding the therapeutic agent and the polyphenol-containing composition.
Clause 23. The dosage form of any one of clauses 1-21, in the form of a gastrointestinal dosage form, such as a suppository.
Clause 24. A method of delivering an active agent to a patient, comprising:
delivering to the intestine of a patient, e.g., enterically, an amount of a polyphenol-containing composition comprising a polyphenol or a mixture of plant polyphenols able to increase permeability of the intestinal epithelium in a patient, in an amount effective to increase permeability of intestinal epithelium of a patient; and
delivering a therapeutic agent ranging in size from 500 Da (Daltons, or 1 kDa) to 150,000 Da, from 1,000 Da to 100,000 Da, from 1,500 Da to 80,000 Da, or from 2,000 Da to 50,000 Da to the intestine at the same time as the delivery of the polyphenol-containing composition to the patient, within 6, 5, 4, 3, 2, or 1 hour, or 30 minutes of delivery of the polyphenol-containing composition to the patient, or during a time period where the permeability of the intestinal epithelium of the patient is increased due to the presence of the polyphenol-containing composition.
Clause 25. The method of clause 24, wherein the polyphenol-containing composition is prepared from a red-pigmented plant material, a pelargonidin-containing plant material, a petunidin-containing plant material, or a malvidin-containing plant material.
Clause 26. The method of clause 24, wherein the red-pigmented plant material is strawberry fruit, red grape skin, red tomato, red potato, or a geranium flower.
Clause 27. The method of any one of clauses 24-26, wherein the polyphenol-containing composition is liquidized or crushed fruit, fruit skin, or flower.
Clause 28. The method of clause 24, wherein the polyphenol-containing composition is dried (e.g., lyophilized) and optionally comminuted (e.g., powdered, chopped, macerated, etc.) fruit, fruit skin, or flower.
Clause 29. The method of clause 24, wherein the polyphenol-containing composition is a polyphenol extract of a fruit, a fruit skin, or a flower.
Clause 30. The method of any one of clauses 24-29, wherein the polyphenol-containing composition comprises one or more flavonoids or anthocyanidins or anthocyanins.
Clause 31. The method of clause 30, wherein the polyphenol-containing composition is an anthocyanidin-enriched or anthocyanin-enriched fraction prepared from a fruit, a fruit skin, or a flower.
Clause 32. The method of clause 24, wherein the polyphenol-containing composition comprises pelargonidin or a glycoside thereof, such as callistephin.
Clause 33. The method of clause 24, wherein the polyphenol-containing composition comprises petunidin or a glycoside thereof, such as petunidin-3-O-glucoside.
Clause 34. The method of clause 24, wherein the polyphenol-containing composition comprises malvidin or a glycoside thereof, such as oenin.
Clause 35. The method of clause 24, wherein the polyphenol-containing composition is a fraction prepared from a fruit, fruit skin, or flower that is retained on a hydrophobic resin in water and is eluted from the resin in ethanol.
Clause 36. The method of clause 24, wherein the hydrophobic resin is a macroreticular aliphatic crosslinked polymer composition, such as an acrylic polymer composition.
Clause 37. The method of any one of clauses 24-36, wherein the therapeutic agent is insulin.
Clause 38. The method of clause 37, wherein the insulin is provided in an amount ranging from 5 IU insulin to 200 IU insulin per unit dose.
Clause 39. The method of any one of clauses 24-36, wherein the therapeutic agent is a protein.
Clause 40. The method of any one of clauses 24-36, wherein the therapeutic agent is one or more of abaloparatide, adrenocorticotropic hormone, afamelanotide, albiglutide, ambamustine, atosiban, aviptadil, buserelin, carbetocin, carfilzomib, carperitide, cetrorelix, cholecystokinin, calcitonin (salmon or human), carperitide, corticotropin, cyclosporine, degarelix, desmopressin, dulaglutide, elcatonin, eledoisin, enalapril, enfuvirtide, etelcalcetide, exenatide, felypressin, ganirelix, glatiramer, glucagon, glucagon-like peptide 2, glucose-dependent insulinotropic peptide, gonadorelin, goserelin, heparin, histrelin, human growth hormone, icatibant, lanreotide, leuprorelin (leuprolide), linaclotide, liraglutide, lisinopril, lixisenatide, lucinactant, lutetium, lypressin, mifamurtide, nafarelin, nesiritide, octreotide, ornipressin, oxytocin, pasireotide, plecanatide, pramlintide, romiplostim, romurtide, somatostatin, taltirelin, teduglutide, teriparatide, terlipressin, tetracosactide, thymopentin, triptorelin, vasopression, virus capsid proteins and other antigens, voclosporin, or ziconotide. Specific active ingredients in the binding agents class deliverable by the methods, dosage forms, or compositions described herein include, without limitation: abciximab, adalimumab, alefacept, alemtuzumab, alirocumab, altumomab pentetate, arcitumomab, atezolizumab, avelumab, basiliximab, bectumomab, belimumab, benralizumab, bermekimab, besilesomab, bevacizumab, bezlotoxumab, biciromab, blinatumomab, blontuvetmab, brentuximab vedotin, brodalumab, burosumab, canakinumab, caplacizumab, capromab pendetide, removab, cemiplimab, certolizumab pegol, cetuximab, clivatuzumab tetraxetan, cosfroviximab, daclizumab, daratumumab, denosumab, dinutuximab, dupilumab, durvalumab, eculizumab, edrecolomab, efalizumab, elotuzumab, emapalumab, emicizumab, erunumab, ertumaxomab, etanercept, etracizumab, evolocumab, famolesomab, fantolizumab, fremanezumab, galcanezumab, gemtuzumab ozogamicin, girentuximab, golimumab, guselkumab, ibalizumab, ibritumomab tiuxetan, idarucizamab, igovomab, imciromab, ipilimumab, itolizumab, ixekizumab, labetuzumab, lanadelumab, loviketmab, mepolizumab, mogamulizumab, motavizumab, muromonab-CD3, naptumomab estefenatox, natalizumab, necitumumab, nimotuzumab, nivolumab, nofetumomab, oblitoxaximab, obinutuzumab, ocrelizumab, ofatumumab, olaratumab, omalizumab, oportuzumab, oregovomab, palivizumab, panitumumab, pembrolizumab, pemtumomab, pertuzumab, racotumomab, ramucirumab, ranibizumab, ravulizumab, reslizumab, risankizumab, rituximab, romosozumab, rovelizumab, ruplizumab, sarilumab, secukinumab, siltuximab, sulesomab, tamtuvetmab, tefibazumab, tildrakizumab, toclizumab, tositumomab, trastuzumab, ustekinumab, vedolizumab, visilizumab, votumumab, zalutumumab, or zanolimumab.
Clause 41. The method of clause 40, wherein the therapeutic agent is a binding reagent, such as an antibody fragment.
Clause 42. The method of clause 41, wherein the therapeutic agent is an F(ab) fragment, an F(ab′)₂ fragment, an F(ab′) fragment, an scFv (single-chain variable fragment), a di-scFv (dimeric single-chain variable fragment), a bi-specific T-cell engager (BiTE), a single-domain antibody (sdAb), or an antibody binding domain.
Clause 43. The method of any one of clauses 24-36, wherein the therapeutic agent is a carbohydrate such as a polysaccharide or a glycosaminoglycan.
Clause 44. The method of any one of clauses 24-36, wherein the therapeutic agent is heparin.
Clause 45. The method of any one of clauses 24-44, wherein the therapeutic agent and the polyphenol-containing composition are contained within an oral, enteric-coated dosage form
Clause 46. The method of any one of clauses 24-44, wherein the therapeutic agent and the polyphenol-containing composition are contained within a gastrointestinal dosage form.
Clause 47. A method of reversibly increasing intestinal epithelium permeability in a patient in need thereof, comprising, delivering to an intestine of a patient an amount of a polyphenol-containing composition comprising a polyphenol or a mixture of plant polyphenols able to increase permeability of the intestinal epithelium in a patient, in an amount effective to increase permeability of intestinal epithelium of a patient, wherein the polyphenol-containing composition comprises:
isolated pelargonidin;
a pelargonidin-enriched fraction obtained from a natural plant material;
a mixture of isolated or enriched polyphenols comprising pelargonidin;
isolated petunidin;
a petunidin-enriched fraction obtained from a natural plant material;
a mixture of isolated or enriched polyphenols comprising petunidin;
isolated malvidin;
a malvidin-enriched fraction obtained from a natural plant material;
a mixture of isolated or enriched polyphenols comprising malvidin; or
any combination thereof.
Clause 48. A method of treating hyperglycemia or diabetes in a patient, comprising administering an amount of the dosage form of clause 14 to the patient effective to treat hyperglycemia or diabetes in a patient.

### BRIEF DESCRIPTION OF THE DRAWINGS

FIG. 1. FIG. 1 illustrates an example of a food-derived permeation enhancer for oral macromolecule delivery. Oral delivery of most therapeutics requires transport across the intestinal epithelium. Normally, macromolecule drugs (e.g. therapeutic proteins and nucleic acids) do not pass through the layer of epithelial cells and into the blood stream without an absorption enhancer. Here, Procyanidin B1, a molecule derived from strawberries causes permeability increases in intestinal epithelia (shown larger for clarity) opens tight junctions to allow absorption of large therapeutics without breaching the epithelial barrier to bacteria.

FIG. 2. FIG. 2 displays the relationships between cytotoxicity and permeability changes in Caco-2 intestinal monolayers treated with food extracts. Of approximately fifty extracts to undergo full screening at a treatment concentration of 15 mg/ml, the majority did not significantly affect diffusive resistance across the epithelia (measured by TEER) or cell viability. Several samples permeabilized the monolayers to varying degrees without damaging the cells, while a handful of samples significantly decreased permeability. Finally, a small number of food extracts did exhibit cell toxicity, likely due to the treatment concentration being much larger than that of foodstuffs normally found in the intestines. Error bars represent s.e.m. (n=8 for toxicity and n=3 for TEER).

FIG. 3. FIG. 3 shows selected results of TEER trials for the six of the most effective permeation-modulating food extracts, tested at 15 mg/ml. Culinary aloe, almost completely eradicated resistance of the Caco-2 monolayers, but this effect was not reversible and the cells did not recover their barrier function after 24 hours. Strawberry and red grape reversibly increased monolayer permeability, with strawberry being identified as the most effective reversible permeation enhancer. By contrast, blueberry, orange, and raspberry all decreased monolayer permeability, with raspberry providing the greatest increase in TEER values. Error bars represent s.e.m. (n=3).

FIG. 4. FIG. 4 shows changes in Caco-2 monolayer permeability to the paracellular diffusion marker calcein as a result of treatment with food extracts (15 mg/ml) As in the TEER trials, strawberry was shown to be the most effective of the reversible permeation enhancing foods, while monolayers that were completely permeabilized by aloe did not fully recover their barrier function after the treatment was removed. As predicted by TEER results, the orange, blueberry, and raspberry extracts reduced calcein passage through the epithelial model. Error bars represent s.e.m. (n=3).

FIG. 5. FIG. 5 displays a separation scheme for preliminarily isolating active, permeation enhancing chemicals from strawberry crude extracts. First, lyophilized strawberry material is ground extracted with 80% aqueous ethanol for 24 hours. The resulting material is dried via rotary evaporation and lyophilization, then dissolved in methanol and adsorbed onto Amberlite™ XAD 7 HP (acrylate ester) resin. The methanol was then removed from the resin via filtration and evaporated to dryness, yielding unabsorbed material, which comprises a wide variety of chemistries (MeOH material). The beads were washed twice with deionized water, which was lyophilized to produce a sample composed primarily of sugars and organic acids (H₂O material). Next, the beads were washed with ethanol to collect the remainder of the adsorbed material. The ethanol was removed via rotary evaporation to yield the polyphenolic fraction.

FIG. 6. FIG. 6 shows the relative efficacies of different strawberry fractions. Strawberry polyphenols potently increased paracellular permeability of Caco-2 monolayers to calcein with approximately the same efficacy as the crude strawberry extract when administered at only 33% of the dose by mass. Error bars represent s.e.m., n=3. *p<0.05 w.r.t. control.

FIG. 7. FIG. 7 displays some polyphenols that may be responsible for strawberries' permeation enhancing effects. Polyphenolic compounds consist of at least two linked aromatic rings, with at least one hydroxyl group attached to each ring. Flavonoids, an abundant class of polyphenolics in many fruits, consist of three rings; two aromatic C6 rings, and one 6-member oxygen heterocycle. (a) Pelargonidin is a member of the anthocyanidin class of flavonoids, and with its glycosides provides most of the red color in strawberries. (b) Linking two or more flavonoid units together by carbon-carbon linkages yields members of the condensed tannin class. Procyanidin B1 is an example of a condensed tannin dimer commonly found in strawberries, (c) but these molecules can reach degrees of polymerization upwards of 30 in some fruits.

FIG. 8. FIG. 8 demonstrates strawberry's ability to increase intestinal absorption of macromolecules in mice. (a) Mice treated orally with strawberry extract absorbed more than twice as much FITC-DX4 (a 4 kDa model drug also delivered orally) into their blood circulation. (b) Strawberry treatment likewise improved the absorption of orally delivered FITC-DX40 (a 40 kDa macromolecule) by more than 100%. Error bars display s.e.m. (n=6).

FIG. 9: Graph showing that color of the crude extracts predicted permeation enhancing efficacy for some foods. Calcein permeability through monolayers treated with three different types of fruits and vegetables indicated that only those varieties with red coloration were effective permeation enhancers. All monolayers recovered their barrier function after treatment, with the exception of those exposed to red potato extract. Error bars represent s.e.m., *p<0.05 w.r.t. control.

FIG. 10: Graphs demonstrating that strawberry polyphenols enable absorption of insulin across the intestinal lining. One hour following oral gavage of strawberry polyphenols to mice, an intestinal injection of 1 U/kg insulin induced sustained reductions in blood glucose levels (left). A 1 U/kg subcutaneous insulin dose induced a pronounced but brief response. Integrated areas above the curves (right) from show that oral delivery with STRB PPh resulted in approximately twice the total pharmacodynamic effect as subcutaneous injection. Error bars represent s.e.m., n=5, *p<0.05 w.r.t. control.

FIG. 11: Scheme detailing the process of fractionating strawberry polyphenols via medium pressure liquid chromatography (MPLC). Two tiers of MPLC were used to separate the STRB PPh extract into fractions. The first run, α, yielded three fractions for direct biological testing (α1-α3) and four sets of material for the second tier of MPLC runs. These runs yielded the β, γ, δ, and ε fractions, as well as pure compounds η and θ.

FIG. 12: Graph demonstrating that only one strawberry polyphenol fraction and one purchased polyphenol reference standard act as permeation enhancers. When screened for activity on Caco-2 monolayers, only the ε3 fraction was an effective permeation enhancer. Similarly, phenolic compounds known to occur in strawberries, only the pigment molecule pelargonidin significantly increased the permeability of calcein across cell monolayers. Error bars represent s.e.m., n=3 for calcein experiments.

FIG. 13: Graphs displaying evidence that the effective strawberry polyphenol fraction and the effective compound, pelargonidin, are chemically identical. UPLC traces for fraction ε3 and pelargonidin contain the same characteristic peaks in absorbance (at 280 nm) at approximately 2.1 and 3.3 minutes (left graphs) as well as similar absorption spectra (right graphs), indicating that they are likely chemically identical.

FIG. 14: Graphs demonstrating that pelargonidin is a reversible, efficacious permeation enhancer in cell culture. By TEER, pelargonidin gave a dose-dependent opening of the tight junctions between 0.33 and 1.00 mg/mL. However, only the two lower concentrations recovered their barrier function within 24 hours. The dose dependence was also evident in pelargonidin's improvement of calcein permeability through the monolayers. Error bars display s.e.m., n=3.

FIG. 15: Graph showing that pelargonidin is an effective permeation enhancer for oral delivery of large molecules. Treatment with pelargonidin doubled oral uptake of 4 kDa dextran, a model macromolecular drug, when compared to mice receiving just saline control. Error bars represent s.e.m., n=5, *p<0.05

FIG. 16: Graphs demonstrating that pelargonidin enables absorption of insulin across the intestinal lining. One hour following oral gavage of pelargonidin to mice, an intestinal injection of 1 U/kg insulin induced sustained reductions in blood glucose levels (left). Integrated areas above the curves (right) from show that oral delivery with pelargonidin is approximately 1.3 times the total pharmacodynamic effect of subcutaneous injection. Error bars represent s.e.m., n=5, *p<0.05 w.r.t. control.

FIG. 17: Graph demonstrating that anthocyanidins are efficacious permeation enhancers in cell culture. Of the six anthocyanidins commonly found in fruits and vegetables, applied at 0.33 mg/ml, pelargonidin, petunidin, and malvidin result in statistically significant increases in intestinal epithelial permeability. Error bars display s.e.m., n=3. *p<0.05 w.r.t. untreated control.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions also refer to word forms, cognates and grammatical variants of those words or phrases.

As used herein, the terms “comprising,” “comprise” or “comprised,” and variations thereof, in reference to elements of an item, composition, apparatus, method, process, system, claim etc. are intended to be open-ended, meaning that the item, composition, apparatus, method, process, system, claim etc. includes those elements and other elements can be included and still fall within the scope/definition of the described item, composition, apparatus, method, process, system, claim etc. As used herein, “a” or “an” means one or more. As used herein “another” may mean at least a second or more.

As used herein, the terms “patient” or “subject” refer to members of the animal kingdom, including, but not limited to human beings.

Provided herein is a dosage form, e.g., an oral dosage form, for delivery of high molecular weight therapeutic agents, to the intestine, e.g., the small intestine (enterically), of a patient. In aspects, the therapeutic agent has a molecular weight of at least e.g., greater than 10 Da (Dalton), e.g., having a molecular weight ranging in size from 10 Da to 150,000 Da (150 kDa, or kiloDaltons), from 10 Da to 120 kDa, such as, from 100 Da to 110 kDa, from 100 Da to 80 kDa, from 100 Da to 40 kDa, from 100 Da to 25 kDa, or from 100 Da to 10 kDa, from 1,000 Da to 100,000 Da, from 1,500 Da to 80,000 Da, or from 2,000 Da to 50,000 Da. The dosage form comprises the therapeutic agent and a polyphenol-containing composition, e.g., an anthocyanidin-containing composition, comprising a polyphenol or a mixture of plant polyphenols able to increase permeability of the intestinal epithelium in a patient, and in an amount effective to increase permeability of intestinal epithelium of a patient. The dosage form may comprise a delayed-release coating, such as an enteric coating surrounding the therapeutic agent and the polyphenol-containing composition and delaying release of the therapeutic agent until it reaches the small intestine. The polyphenol-containing composition may comprise pelargonidin, petunidin, or malvidin, or any combination thereof.

Also provided herein is a method of delivering a high molecular weight drug to a patient. The method comprises enterically-delivering (administering) to the patient (that is, administering to a patient's small intestine, by mouth or otherwise), or otherwise administering to a patient's gastrointestinal tract, such as an amount of a polyphenol-containing composition comprising a polyphenol or a mixture of plant polyphenols able to increase permeability of the intestinal epithelium in a patient, concurrently with, or followed by enterically-delivering a therapeutic agent ranging in size from 500 Da (Daltons, or 1 kDa) to 150,000 Da, from 1,000 Da to 100,000 Da, or from 2,000 Da to 80,000 Da to the patient. The therapeutic agent may be enterically-delivered at the same time as the enteric delivery of the polyphenol-containing composition to the patient, or during a time period where the permeability of the intestinal epithelium of the patient is increased due to the presence of the polyphenol-containing composition polyphenol-containing composition in the patient's intestine, e.g., within 6, 5, 4, 3, 2, or 1 hour, or 30 minutes of enteric delivery of the polyphenol-containing composition to the patient.

An “effective amount” or “amount effective” to achieve a desirable therapeutic, pharmacological, medicinal, or physiological effect is any amount that achieves the stated purpose. For example, an amount of the polyphenol-containing composition effective to increase permeability of intestinal epithelium of a patient and/or effectively deliver bioactive amounts of a therapeutic agent through the intestinal epithelium. Based on the teachings provided herein, one of ordinary skill can readily ascertain effective amounts of the elements of the described dosage form and produce a safe and effective dosage form and drug product.

A therapeutic agent is any compound or composition that is delivered to a patient to achieve a desired effect, such as a beneficial, treatment, or curative effect. Therapeutic agents include proteins, such as polypeptides or proteins. In the context of the present invention, therapeutic agents of high molecular weight, e.g., greater than 1 kDa, e.g., having a molecular weight ranging in size from 500 Da to 150,000 Da, from 1,000 Da to 100,000 Da, or from 1,500 Da to 80,000 Da, or from 2,000 Da to 50,000 Da is deliverable by the method and dosage form described herein. Within this class of therapeutic agents are many compounds or compositions that can be described as “biologicals”. Many therapeutic agents within this class are either approved for marketing or are being evaluated for use in humans or animals, such as, for example and without limitation, binding reagents, interleukins, cytokines, hormones, growth factors, supplements, and other compositions of either natural or synthetic/recombinant origin. In one example, the therapeutic agent is insulin. In another, it is a binding reagent such as IgG antibody for immunotherapy. In a third example, the therapeutic agent is a carbohydrate or polysaccharide, including glycosaminoglycans, such as heparin, and heparin derivatives.

In terms of molecular weight of compounds deliverable by the method and dosage form described herein, the composition and physical qualities of the therapeutic agent can vary greatly and as such there will be some expected variation in the molecular weight of the deliverable therapeutic agent due to, for example and without limitation: size, secondary structure, hydrophobicity, flexibility, and composition of the therapeutic agent. As illustrated in the Examples below, the dosage form and method provided herein can enhance intestinal, e.g., enteric, delivery of macromolecules with significantly-varying composition and size. It should be noted that for polydisperse polymers, unless specified otherwise, the molecular weight refers to the number average molecular weight (Mn) of the polymer composition).

In further detail, the size of the active ingredient delivered by the methods, compositions, and dosage forms described herein is relevant to the ability of that active ingredient to permeate epithelial tissue. In one aspect, the active ingredient (e.g., drug) is less than 120 kDa (kiloDaltons, either in molecular weight or, in the case of polydisperse compounds, number average molecular weight), e.g., ranging from 10 Da to less than 120 kDa, such as from 100 Da to 110 kDa, from 100 Da to 80 kDa, from 100 Da to 40 kDa, from 100 Da to 25 kDa, or from 100 Da to 10 kDa.

The active ingredient may be, without limitation, one or more of an antiseptic, an antibiotic, an analgesic, an anesthetic, a chemotherapeutic agent, a clotting agent, an anti-inflammatory agent, a cytokine, a hormone, a steroid, a protein, or a nucleic acid. Active ingredients that may be incorporated, by themselves, or in combination with a suitable excipient, into any composition, device, dosage form, or drug product described herein include, without limitation: anti-inflammatories, such as, without limitation, NSAIDs (non-steroidal anti-inflammatory drugs) such as salicylic acid, indomethacin, sodium indomethacin trihydrate, salicylamide, naproxen, colchicine, fenoprofen, sulindac, diflunisal, diclofenac, indoprofen sodium, salicylamide, anti-inflammatory cytokines, and anti-inflammatory proteins or steroidal anti-inflammatory agents; antibiotics and antivirals, such as, without limitation: acyclovir, afloxacin, ampicillin, amphotericin B, atovaquone, azithromycin, ciprofloxacin, clarithromycin, clindamycin, clofazimine, dapsone, diclazaril, doxycycline, erythromycin, ethambutol, fluconazole, fluoroquinolones, foscarnet, ganciclovir, gentamicin, iatroconazole, isoniazid, ketoconazole, levofloxacin, lincomycin, miconazole, neomycin, norfloxacin, ofloxacin, paromomycin, penicillin, pentamidine, polymixin B, pyrazinamide, pyrimethamine, rifabutin, rifampin, sparfloxacin, streptomycin, sulfadiazine, tetracycline, tobramycin, trifluorouridine, trimethoprim sulphate, Zn-pyrithione, ciprofloxacin, norfloxacin, afloxacin, levofloxacin, gentamicin, tobramycin, neomycin, erythromycin, trimethoprim sulphate, polymixin B and silver salts such as chloride, bromide, iodide and periodate; anticlotting factors such as heparin, Pebax, enoxaprin, aspirin, hirudin, bivalirudin, prasugrel, idraparinux, warfarin, clopidogrel, PPACK, GGACK, tissue plasminogen activator, urokinase, and streptokinase; growth factors; immunosuppressants; glucocorticoids such as hydrocortisone, betamethasone, dexamethasone, flumethasone, isoflupredone, methylpred-nisolone, prednisone, prednisolone, and triamcinolone acetonide; antiangiogenics, such as fluorouracil, paclitaxel, doxorubicin, cisplatin, methotrexate, cyclophosphamide, etoposide, pegaptanib, lucentis, tryptophanyl-tRNA synthetase, anecortave, CA4P, AdPEDF, VEGF-TRAP-EYE, Avastin, JSM6427, TG100801, ATG3, OT-551, endostatin, thalidomide, becacizumab, neovastat; antiproliferatives such as sirolimus, paclitaxel, perillyl alcohol, farnesyl transferase inhibitors, FPTIII, L744, antiproliferative factor, 5-FU, Daunomycin, Mitomycin, dexamethasone, azathioprine, chlorambucil, methotrexate, mofetil, vasoactive intestinal polypeptide, and PACAP; antibodies and fragments thereof; antigens for vaccinations, including virus capsid proteins and fragments thereof; peptide hormones, such as amylin, angiotensin, calcitonin, endothelin, glucagon, glucagon-like-peptide-1, human chorionic gonadotropin, human placental lactogen, growth hormone, insulin, insulin-like growth factor, luteinizing hormone, oxytocin, parathyroid hormone, prolactin, relaxin, secretin, thyroid-stimulating hormone.

Examples of active ingredients (e.g., drugs) that are presumed to be deliverable by the methods, compositions, and drug products described herein, include, without limitation: biologics; proteins; peptides; nucleic acids, including nucleic acid analogs such as DNA, RNA, peptide nucleic acid (PNA), short interfering RNA (siRNA), messenger RNA (mRNA), microRNA (miRNA), tRNA, phosphorothioate, locked nucleic acid, unlocked nucleic acid, 2′-O-methyl-substituted RNA, morpholino nucleic acid, threose nucleic acid, glycol nucleic acid backbone, or modified RNA bases, including pseudouridine, 1-methylpseudouridine, 5-methylcytidine, or 2-thiouridine, or any combination thereof, including aptamers, as are broadly-known. Nucleic acid analogs also include peptide nucleic acids, such as γ-peptide nucleic acids. As indicated above, the active ingredients have a molecular weight (for a defined compound, including defined proteins, peptides, nucleic acids, or aptamers, or a number average molecular weight Mn for a polydisperse compound or composition, such as a polymer or a polymer-modified protein, peptide, aptamer or small molecule) of less than 120 kDa, 80 kDa, 40 kDa, 25 kDa, 10 kDa, or increments therebetween, and no effective minimal molecular weight, though greater than 1 Da, and more realistically, greater than 50 Da or 100 Da.

Specific active ingredients in the protein and peptide class deliverable by the methods, dosage forms, or compositions described herein include, without limitation: abaloparatide, adrenocorticotropic hormone, afamelanotide, albiglutide, ambamustine, atosiban, aviptadil, buserelin, carbetocin, carfilzomib, carperitide, cetrorelix, cholecystokinin, calcitonin (salmon or human), carperitide, corticotropin, cyclosporine, degarelix, desmopressin, dulaglutide, elcatonin, eledoisin, enalapril, enfuvirtide, etelcalcetide, exenatide, felypressin, ganirelix, glatiramer, glucagon, glucagon-like peptide 2, glucose-dependent insulinotropic peptide, gonadorelin, goserelin, heparin, histrelin, human growth hormone, icatibant, insulin, lanreotide, leuprorelin (leuprolide), linaclotide, liraglutide, lisinopril, lixisenatide, lucinactant, lutetium, lypressin, mifamurtide, nafarelin, nesiritide, octreotide, ornipressin, oxytocin, pasireotide, plecanatide, pramlintide, romiplostim, romurtide, somatostatin, taltirelin, teduglutide, teriparatide, terlipressin, tetracosactide, thymopentin, triptorelin, vasopression, virus capsid proteins and other antigens, voclosporin, or ziconotide. Specific active ingredients in the binding agents class deliverable by the methods, dosage forms, or compositions described herein include, without limitation: abciximab, adalimumab, alefacept, alemtuzumab, alirocumab, altumomab pentetate, arcitumomab, atezolizumab, avelumab, basiliximab, bectumomab, belimumab, benralizumab, bermekimab, besilesomab, bevacizumab, bezlotoxumab, biciromab, blinatumomab, blontuvetmab, brentuximab vedotin, brodalumab, burosumab, canakinumab, caplacizumab, capromab pendetide, removab, cemiplimab, certolizumab pegol, cetuximab, clivatuzumab tetraxetan, cosfroviximab, daclizumab, daratumumab, denosumab, dinutuximab, dupilumab, durvalumab, eculizumab, edrecolomab, efalizumab, elotuzumab, emapalumab, emicizumab, erunumab, ertumaxomab, etanercept, etracizumab, evolocumab, famolesomab, fantolizumab, fremanezumab, galcanezumab, gemtuzumab ozogamicin, girentuximab, golimumab, guselkumab, ibalizumab, ibritumomab tiuxetan, idarucizamab, igovomab, imciromab, ipilimumab, itolizumab, ixekizumab, labetuzumab, lanadelumab, loviketmab, mepolizumab, mogamulizumab, motavizumab, muromonab-CD3, naptumomab estefenatox, natalizumab, necitumumab, nimotuzumab, nivolumab, nofetumomab, oblitoxaximab, obinutuzumab, ocrelizumab, ofatumumab, olaratumab, omalizumab, oportuzumab, oregovomab, palivizumab, panitumumab, pembrolizumab, pemtumomab, pertuzumab, racotumomab, ramucirumab, ranibizumab, ravulizumab, reslizumab, risankizumab, rituximab, romosozumab, rovelizumab, ruplizumab, sarilumab, secukinumab, siltuximab, sulesomab, tamtuvetmab, tefibazumab, tildrakizumab, toclizumab, tositumomab, trastuzumab, ustekinumab, vedolizumab, visilizumab, votumumab, zalutumumab, or zanolimumab.

As used herein, the term “binding reagent” refers to a molecule capable of interacting with a target molecule. Binding reagents having limited cross-reactivity are generally preferred. In certain embodiments, suitable binding reagents include, for example, polypeptides, such as for example, antibodies, monoclonal antibodies, or derivatives or analogs thereof, including without limitation: Fv fragments, single chain Fv (scFv) fragments, Fab₁ fragments, F(ab′)₂ fragments, single domain antibodies, camelized antibodies and antibody fragments, humanized antibodies and antibody fragments, and multivalent versions of the foregoing; multivalent binding reagents including without limitation: monospecific or bispecific antibodies, such as disulfide stabilized Fv fragments, scFv tandems ((ScFv)₂ fragments), diabodies, triabodies, tetrabodies, which typically are covalently linked or otherwise stabilized (i. e., leucine zipper or helix stabilized) scFv fragments, di-scFv (dimeric single-chain variable fragment), single-domain antibody (sdAb), or antibody binding domain fragments and other binding reagents including, for example, bi-specific T-cell engagers (BiTEs), aptamers, template imprinted materials (such as those of U.S. Pat. No. 6,131,580), and organic or inorganic binding elements. In exemplary embodiments, a binding reagent specifically interacts with a single epitope. In other embodiments, a binding reagent may interact with several structurally related epitopes.

The term “ligand” refers to a binding moiety for a specific target, its binding partner. The molecule can be a cognate receptor, a protein a small molecule, a hapten, or any other relevant molecule. The term “antibody” refers to an immunoglobulin, derivatives thereof which maintain specific binding ability, and proteins having a binding domain which is homologous or largely homologous to an immunoglobulin binding domain. As such, the antibody operates as a ligand for its cognate antigen, which can be virtually any molecule. Natural antibodies comprise two heavy chains and two light chains and are bi-valent. The interaction between the variable regions of heavy and light chain forms a binding site capable of specifically binding an antigen (e.g., a paratope). The term “VH” refers to a heavy chain variable region of an antibody. The term “VL” refers to a light chain variable region of an antibody. Antibodies may be derived from natural sources, or partly or wholly synthetically produced. An antibody may be monoclonal or polyclonal. The antibody may be a member of any immunoglobulin class, including any of the human classes: IgG, IgM, IgA, IgD, and IgE.

The term “antibody fragment” refers to any derivative of an antibody which is less than full-length. In exemplary embodiments, the antibody fragment retains at least a significant portion of the full-length antibody's specific binding ability. Examples of binding reagents, including antibody fragments, but are not limited to, Fab, Fab′, F(ab′)₂, Fv, Fd, dsFv, scFv, diabody, triabody, tetrabody, di-scFv (dimeric single-chain variable fragment), bi-specific T-cell engager (BiTE), single-domain antibody (sdAb), or antibody binding domain fragments. The antibody fragment may be produced by any means. For instance, the antibody fragment may be enzymatically or chemically produced by fragmentation of an intact antibody, or it may be recombinantly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

Antibody fragments also include miniaturized antibodies or other engineered binding reagents that exploit the modular nature of antibody structure, comprising, often as a single chain, one or more antigen-binding or epitope-binding sequences and at a minimum any other amino acid sequences needed to ensure appropriate specificity, delivery, and stability of the composition (see, e.g., Nelson, A L, “Antibody Fragments Hope and Hype” (2010) MAbs 2(1):77-83).

In certain embodiments, the binding reagent is an antibody or an antibody fragment. In another embodiment, the binding reagent has a molecular weight of less than 150 kDa, 125 kDa, or 100 kDa, or less than or equal to 80 kDa. For example, the binding reagent may be a monoclonal antibody, or derivatives or analogs thereof, including without limitation: Fv fragments, single chain Fv (scFv) fragments, Fab′ fragments, F(ab′)2 fragments, single domain antibodies, camelized antibodies and antibody fragments, humanized antibodies and antibody fragments, and multivalent versions of the foregoing; multivalent activators including without limitation: monospecific or bispecific antibodies, such as disulfide stabilized Fv fragments, scFv tandems ((scFv)2 fragments), diabodies, tribodies or tetrabodies, which typically are covalently linked or otherwise stabilized (i.e., leucine zipper or helix stabilized) scFv fragments; receptor molecules which naturally interact with a desired target molecule.

A carbohydrate is an organic molecule comprising carbon, oxygen, and hydrogen. A polysaccharide is a polymeric carbohydrate comprising chains of three or more monosaccharide units bound together by glycoside linkages, and includes glycosaminoglycans (GAGs) or mucopolysaccharides. Polysaccharides deliverable by the methods and dosage forms provided herein include heparin, heparin sulfate, and inulin. Heparin is a commonly-used anticoagulant, and is administered therapeutically in parenteral form. As a class, “heparin” includes pharmacologically active derivatives thereof, e.g., as are broadly-known in the art.

“Insulin” refers to a pharmacologically active insulin composition that includes as a class human insulin and human insulin analogs (insulin equivalents) that are acceptable for insulin therapy in a patient, such as a human patient. A number of human insulin analogs are known, and include fast-acting insulin, medium-acting insulin, and long-acting insulin, and include as a class, for example and without limitation, insulin aspart, insulin lispro (lyspro), insulin glulisine, insulin glargine, insulin detemir, insulin degludec, lente insulin, and ultralente insulin, as are broadly-known. Insulin analogs generally have modified amino acid sequences as compared to human insulin, either produced by genetic engineering, or by virtue of being xenogeneic in origin with respect to the end user of the insulin product. For example, insulin glargine comprises an asparagine-to-glycine substitution and two additional C-terminal arginine residues as compared to normal human insulin. As another example, beef insulin (lente) or porcine insulin (NPH insulin, formulated with protamine) have been used in human drugs. Activity of insulin, and the amount of insulin in a drug product is quantified in terms of “units” of activity, that are standardized, though which may change over time. In one example, one international unit (1 IU or U1) and one USP unit of human insulin is the activity contained in, or the biological equivalent of 0.0347 mg of the international standard for human insulin, e.g., crystalline human insulin. An oral unit dose of insulin may comprise an amount effective to treat a patient, such as a diabetic patient, and can range greatly with respect to several factors, including, for example and without limitation, the desired release profile in a patient and a patient's weight. The amount of insulin and the release profile may be titrated for a patient, and for the timing of delivery to the patient. In one example, a unit dose of insulin ranges from 5 IU insulin to 200 IU insulin per unit dose. Insulin may be administered either at regular intervals, regular times, or as-needed, for example, at a certain time before a meal.

In the context of delivery of therapeutic agents, delivery profiles may be varied depending on the desired therapeutic effect. The nature of the present disclosure relates to intestinal delivery of therapeutic agents. As such, the therapeutic agent is at least in part delivered to the intestine. This is accomplished by delayed-release dosage forms, such as enteric or timed-release oral dosage forms. A “dosage form” is the physical form of a dose of a chemical compound used as a drug or medication intended for administration or consumption. A dosage form corresponds to a unit dosage form, even when dispensed from a drug product that includes multiple doses.

The polyphenol-containing composition and the therapeutic agent may be compounded or otherwise manufactured into a suitable composition for use, such as a pharmaceutical dosage form or drug product in which the compound is an active ingredient. Drug products may comprise a pharmaceutically acceptable carrier, or excipient. Therapeutic/pharmaceutical compositions are prepared in accordance with acceptable pharmaceutical procedures, such as described in Remington: The Science and Practice of Pharmacy, 21st edition, ed. Paul Beringer et al., Lippincott, Williams & Wilkins, Baltimore, Md. Easton, Pa. (2005) (see, e.g., Chapters 37, 39, 41, 42 and 45 for examples of powder, liquid, parenteral, intravenous and oral solid formulations and methods of making such formulations). Depending on the delivery route, the dosage form may comprise additional carriers or excipients, such as water, saline (e.g., normal saline), or phosphate-buffered saline, as are broadly-known in the pharmaceutical arts. Compositions may comprise a pharmaceutically acceptable carrier, or excipient. An excipient is an inactive substance used as a carrier for the active ingredients of a medication. Although “inactive,” excipients may facilitate and aid in increasing the delivery, stability or bioavailability of an active ingredient in a drug product. Non-limiting examples of useful excipients include: anti-adherents, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts.

For example, where the dosage form is an oral dosage form, it may comprise a delayed-release coating to delay release of the therapeutic agent and polyphenol-containing composition until it reaches the small intestine. The dosage form may be provided as a unit dosage form, e.g., with the therapeutic agent and polyphenol-containing composition packaged within a syringe, bag, or other suitable packaging for single or multiple use. The dosage form also may be a suitable gastrointestinal dosage form, for example, as a suppository, enema, or through a feeding tube for delivery to the intestine of a patient.

A “delayed-release coating” on a pharmaceutical dosage form is a coating or barrier applied to an oral dosage form that delays release of the active components, e.g., therapeutic agent(s), of the dosage form. A delayed-release coating may be an “enteric coating” that remain intact in the stomach, surviving gastric pH and enzymatic processes, but dissolving in the environment of the small intestine. In the context of the dosage forms described herein, the object of delivery is to delay release of the macromolecular therapeutic agent until it passes into the small intestine. Due to the highly different rates of passage through the stomach depending on gastric content, and varying between individuals, an enteric-release coating or barrier may be preferred over a delayed-release barrier that depends on the passage of time rather than passage into the intestinal environment.

In embodiments, polymers useful for enteric coatings remain intact at low pH, but as the pH increases in the small intestine, the polymer swells or becomes soluble in the intestinal fluid. Non-limiting examples of materials used for enteric coatings include: cellulose acetate phthalate (CAP), poly(methacrylic acid-co-methyl methacrylate), cellulose acetate trimellitate (CAT), poly(vinyl acetate phthalate) (PVAP), and hydroxypropyl methylcellulose phthalate (HPMCP), fatty acids, waxes, shellac, plastics and plant fibers. Delayed release coatings also include polymeric coatings, such as, for example and without limitation, hydroxypropyl methylcellulose (HPMC), carboxymethylcellulose (CMC), or other polymeric compounds can be coated onto a quick-release core comprising a therapeutic agent. The thickness of the coating can be varied to produce a desirable delay in release of the therapeutic agent. Likewise, particles comprising a therapeutic agent and/or the delivery-enhancing polyphenols can be embedded within HPMC, CMC, or other polymers useful in controlling release of a therapeutic agent. The delayed-release coating can be coated with an enteric coating to further control release of the therapeutic agent and delivery-enhancing polyphenols. Those of ordinary skill in the compounding arts can optimize the overall dosage form structure and release patterns in the dosage forms described herein, for any specific therapeutic agent.

Polyphenols are compounds that comprising one or more hydroxyl groups attached to an aromatic ring, and include, as a class hydroxybenzoic acids, hydroxycinnamic acids, flavonoids, stilbenes, and lignans, as are broadly-known (See, e.g., Manach, C., et al., “Polyphenols: food sources and bioavailability” (2004) Am Journal Clin Nutr 79(5):727-747. They abundant in plants, including in the plant bodies, leaves, flowers, and fruits thereof and in that context are referred to as phytochemicals. Flavonoids are substituted flavylium compounds. In flavonoids, one or more H of flavylium (Formula I) is substituted, commonly with hydroxyl (—OH) or methoxyl (—OCH₃) groups.

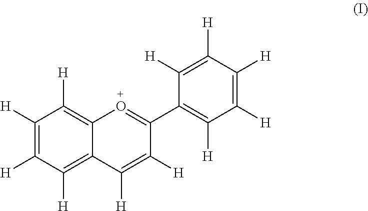

Certain flavonoids, such as anthocyanidins often have the structure of Formula II:

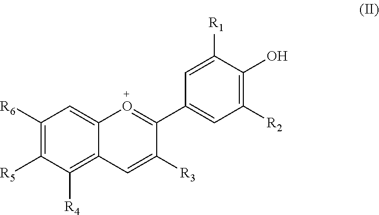

where R₁, R₂, R₃, R₄, R₅, and R₈ are, independently, H, —OH, or —OCH₃. In anthocyanidins, a plant pigment, R₃ is —OH, R₄ is —OH, R₅ is —H, and R₆ is —OH. For example, the compound pelargonidin has the structure of Formula II, where R₁ is H, R₂ is H, R₃ is —OH, R₄ is OH, R₅ is H, and R₆ is —OH:

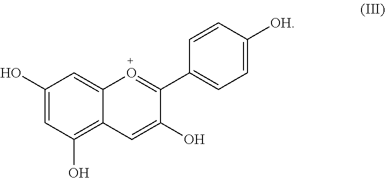

Petunidin has the structure of Formula IV:

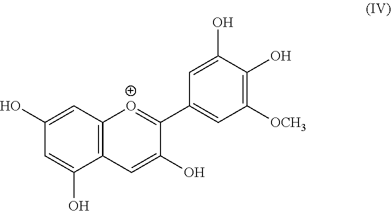 [SMILES: COc1cc(-c2[o+]c3cc(O)cc(O)c3cc2O)cc(O)c1O]

Malvidin has the structure of Formula V:

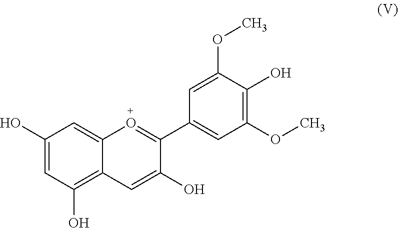

Anthocyanins are sugar-substituted anthocyanidins (glycosides of anthocyanidins). A glycoside of pelargonidin is callistephin (Formula VI). Glycoside of malvidin include, without limitation malvin, oenin, primulin, malvidin 3-rutinosides, and Malvidin-3-O-glucoside-5-O-(6-acetylglucoside). Glycosides of petunidin include, without limitation, petunidin-3-O-glucoside, petunidin-3-O-(6-p-coumaroyl) glucoside, petunidin-3-O-(6-p-acetyl) glucoside, petunidin-3-O-galactoside, and petunidin-3-rutinoside.

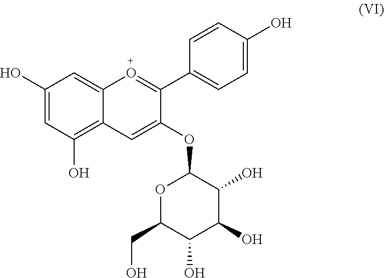

Formulas I-VI, including pelargonidin, petunidin, and malvidin are salts, and are depicted as cations and, as such, “pelargonidin”, “petunidin”, and “malvidin” includes any pharmaceutically-acceptable salt thereof, unless the counterion is specifically stated, as in “pelargonidin chloride”, referring to the chloride salt of pelargonidin. The counterion to produce a pharmaceutically-acceptable salt may be anion, such as, without limitation acetate, aspartate, benzenesulfonate, benzoate, besylate, bicarbonate, bitartrate, bromide, camsylate, carbonate, chloride, citrate, decanoate, edetate, esylate, fumarate, gluceptate, gluconate, glutamate, glycolate, hexanoate, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, acetate, succinate, sulfate, tartrate, teoclate, or tosylate. In one aspect, “pelargonidin”, “petunidin”, and “malvidin” comprise pelargonidin chloride, petunidin chloride, and malvidin chloride, respectively.

Polyphenols, e.g., anthocyanidins and anthocyanins, such as pelargonidin, petunidin, malvidin, or a glycoside of pelargonidin, petunidin, malvidin, may be prepared by any useful method.

The compound(s) may be prepared by a purely synthetic method from suitable precursor(s), or may be isolated from a plant, such as from a fruit, fruit skin, or flower of a plant. A variety of methods have been devised for the fractionation of plant materials to enrich phytochemicals, such as polyphenols, flavonoids, anthocyanidins, and anthocyanins (see, e.g., U.S. Pat. No. 8,987,481; International Patent Application Publication No. WO 2013/086621; Ameer K., et al. “Green Extraction Methods for Polyphenols from Plant Matrices and Their Byproducts: A Review” Compr Rev Food Sci F (2017) 16:295-315; Rajbhar, K., et al., “Polyphenols: Methods of Extraction” (2015) Sci. Revs. Chem. Commun. 5(1), 2015, 1-6; and Martin, J, et al., (Mar. 15, 2017) Anthocyanin Pigments: Importance, Sample Preparation and Extraction, Phenolic Compounds—Natural Sources, Importance and Applications, Marcos Soto-Hernandez, Mariana Palma-Tenango and Maria del Rosario Garcia-Mateos, IntechOpen, DOI: 10.5772/66892).

In the examples below, a polyphenol-enriched, anthocyanin-enriched, and anthocyanidin-enriched fraction of fruit or fruit skin is prepared by grinding of dried plant material followed by extraction in 80% ethanol in water. The ethanol extract is dried and lyophilized, reconstituted in methanol, and absorbed onto a hydrophobic resin, e.g., a macroreticular aliphatic crosslinked polymer composition, such as an acrylic polymer composition, such as Amberlite™ XAD 7 HP. The resin is washed with water, and then retained polyphenols are eluted in ethanol, e.g., 80% ethanol. The ethanol is removed, e.g., by rotary evaporation. The composition can be lyophilized and reconstituted in water or used in a lyophilized state.

Other methods of enriching phytochemicals, such as polyphenols, flavonoids, anthocyanidins, and anthocyanins, include, without limitation, supercritical fluid extraction, ultrasound-assisted extraction, microwave-assisted extraction, pressurized liquid extraction, and pressurized hot water extraction.

As used herein, a “fraction” of a stated composition, such as a fraction of a plant material, is a portion of the plant material separated from other, different, portions by physical, chemical, electrical, or other methods, such as, for example and without limitation, by filtration, adsorption, absorption, affinity, chromatography, centrifugation, electrophoresis, extraction, or any useful means for separating constituents of a mixture of chemically and/or physically different compositions present in plant material. A fraction enriched for a stated constituent includes a greater concentration of that constituent as compared to pre-fractioned material and/or a lesser relative amount of a different constituent as compared to pre-fractioned material. Thus, a polyphenol-enriched fraction of fractionated plant material, such as fruit, fruit skin, or flower, as compared to pre-fractionated plant material, comprises either a greater concentration of a polyphenol composition or a lesser relative amount of one or more non-polyphenol materials, such as non-aromatic compounds or plant solids, as compared to pre-fractioned plant material. Likewise, an anthocyanidin-enriched or an anthocyanin-enriched fraction of fractionated plant material, such as fruit, fruit skin, or flower, as compared to pre-fractionated plant material, comprises either a greater concentration of an anthocyanidin compound or an anthocyanin compound, or a lesser relative amount of one or more other plant materials, such as non-aromatic compounds or plant solids, as compared to pre-fractioned plant material.

Plant materials useful in preparation of the polyphenols of the described drug product and method include strawberries and red grapes. Other red plant materials, for example plant materials or fractions thereof, that comprise pelargonidin, petunidin, malvidin, or other anthocyanidins are expected to be active or are expected to comprise fractions that would be active in reversibly-enhancing intestinal permeability. Such plant materials could include blackberries, apples, tomatoes, red grapes, red potato, and raspberry roots, stems, and leaves.

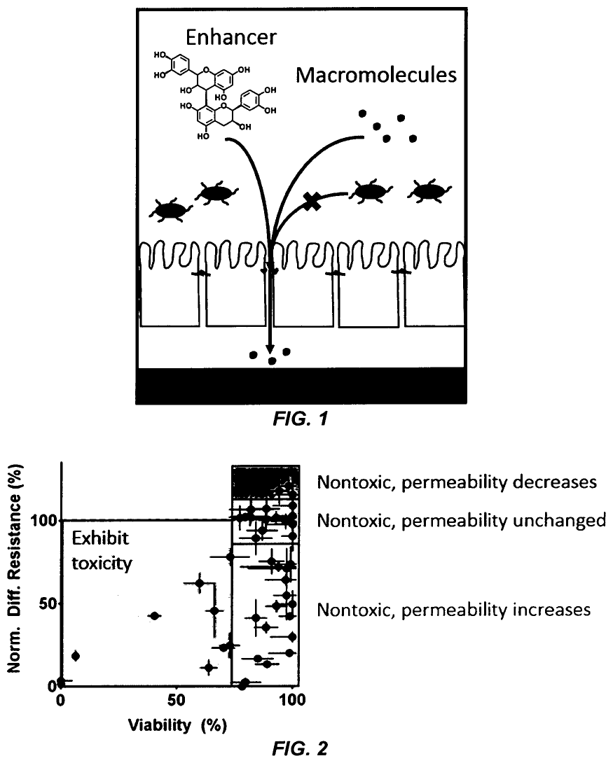

In one aspect of the invention, plant-derived extracts, including fruit-derived extracts, fruit skin-derived extracts, and flower-derived extracts comprising an anthocyanidin, such as pelargonidin, petunidin, or malvidin, or any combination thereof, can reversibly increase the permeability of the intestinal epithelium, and can therefore be used for oral drug delivery. In such a system, it is believed, without any intent to be bound by this theory, that food-derived permeation enhancers act upon the cells and the protein junctions between them, opening pathways for drug macromolecules to diffuse through (FIG. 1) the intestinal epithelium from the intestinal lumen and into tissue of the patient, including the patient's bloodstream.

### EXAMPLES

### Example 1—Identification and Testing of Permeability-Modulating Fractions and Compounds

Materials: Penicillin/streptomycin, trypsin-ethylenediaminetetraacetic acid (trypsin-EDTA), phosphate buffer saline (PBS), fetal bovine serum (FBS), rat tail Collagen I, PrestoBlue® viability kit, and calcein were purchased from Life Technologies® (Thermo Fisher subsidiary, Carlsbad, Calif., USA). Caco-2 cells were purchased from American Type Culture Collection® (ATCC, Manassas, Va., USA). Dulbecco's Modified Eagles Medium (DMEM), Falcon® 225 cm2 tissue culture flasks, Corning® HTS 1.0 μm porous support Transwell® plates, Falcon® 24-well plates, Corning® CellBIND® 96-well microplates, sodium butyrate, MITO+ serum extender, Aimstrip® Plus blood glucose strips, blood glucose monitor, gentisic acid, furoic acid, ellagic acid, kaempferol, naringin, vanillic acid, protocatechuic acid, ferulic acid, caffeic acid, lactone hexose, resveratrol, and luteolin were obtained from VWR® (Radnor, Pa., USA). FITC-labelled dextrans, Amberlite™ XAD7 resin, bovine pancreas insulin, catechin, epicatechin, polydatin, myricitrin, myricetin, hesperetin, myrtenol, and gallic acid were purchased from Sigma-Aldrich® (St. Louis, Mo., USA). C18 bulk silica gel SMT-Bod-C18 was purchased from Seperation Methods Technologies (Newark, Del., USA). Callistephin, procyanidin B1, cyanidin, peonidin, delphinidin, petunidin, and malvidin were obtained from Alkemist Labs (Costa Mesa, Calif., USA). Epicatechin gallate and pelargonidin were from ChromaDex (Irvine, Calif., USA). Genistein, glucogallin, and sarasapogenin were purchased from Toronto Research Chemicals (North York, ON, Canada).

Cell Culture: Caco-2 cells were cultured in DMEM medium supplemented with 10% FBS, 1% Pen/Strep, and 0.1% Amphotericin B (“Caco-2 media”). Cells were passaged every 2 to 4 days at ratios between 1:3 and 1:8.

Preparation of Crude Extracts: Food samples were obtained from local supermarkets. After removing inedible portions, the samples were blended with 125% of weight distilled water on medium-high speed for three minutes using a household blender. The resulting solution was transferred to 50 mL conical tubes and centrifuged for 30 minutes at 1500 RPM. The supernatant was frozen to −80° C. and lyophilized, and the resulting powder stored at −80° C. until use.

PRESTOBLUE® Assay: Caco-2 cells were seeded on a black, 96-well plate at a concentration of 10⁴ cells/well. After incubating the plate overnight at 37° C., the media in the wells was aspirated and replaced with the treatment solutions (100 μL/well). After three hours of exposure, the extracts were aspirated. PrestoBlue® reagent (10 μL/well) and Caco-2 media (100 μL/well) were added to the wells. Thirty minutes later, an automated plate reader was used to measure the fluorescent signal produced by viable cells. The viability of each treatment is expressed as the ratio of the strength of its cells' fluorescent signals to the strength of the untreated cells' fluorescent signals.

Caco-2 Cell Monolayers: Caco-2 cells were seeded in Basal Seeding Medium (BSM) on a collagen-coated, 24-Multiwell Insert TRANSWELL® Plate at a concentration of 2×10⁵ cells per well. The plate was incubated at 37° C. for one to two days. On the third day, the BSM was replaced with Enterocyte Differentiation Medium (EDM). The plate was then incubated one to two more days at 37° C. to allow complete formation of differentiated monolayers.

TEERAssay: All TEER (transepithelial electrical resistance) values were measured using a voltohmmeter. Between each TEER measurement, the plate was incubated at 37° C. for a minimum of fifteen minutes to allow the cells to rest. To ensure that all the wells contained fully formed Caco-2 monolayers (>200 Ω·cm²), initial TEER values were measured prior to treatment exposure. After adding the extracts to each well, TEER values were measured at defined intervals for a total of three hours. The treatments were then aspirated, and the cells rinsed with PBS before refilling the wells with fresh EDM. The plates were then left in the incubator at 37° C. for 24 hours, when recovery TEER values were measured.

Permeability Assays: The paracellular diffusion markers were applied at 0.5 mM (calcein) or 0.1 mM (4 kDa FITC-Dextran), dissolved in EDM with the treatment extracts, to the apical side of fully-formed monolayers (TEER>200 Ω·cm²). Fresh EDM with the markers was used as a negative control. After one hour, media in the basal chambers was sampled and examined for fluorescence at 495/515 nm. Application of calibration curves yielded an amount of mass transferred across each monolayer, which was used in the permeability equation Pₐₚₚ=ΔM/CₐAΔt, where Pₐₚₚ is the apparent permeability through the monolayer, ΔM is the marker mass in the basal compartment, Cₐ is the apical marker concentration, A is the monolayer area, and Δt is the time between samples. Permeability measurements are expressed as the ratio of monolayer permeability at 3 hours after treatment addition to permeability before treatment addition, normalized to any change in negative control monolayers during that time.

AMBERLITE™ Separation of Strawberry Extracts: Strawberry acetone extract was dried via rotary evaporation, dissolved in methanol, and adsorbed onto AMBERLITE™ XAD 7 HP (acrylate ester) resin. The methanol was then removed from the resin and evaporated to dryness, yielding unabsorbed material, which comprises a wide variety of chemistries. The beads were washed with water, which was lyophilized to produce a sample composed primarily of sugars and organic acids. Next, the beads were washed with ethanol to collect the remainder of the adsorbed material. The ethanol was removed via rotary evaporation, and the contents re-suspended in water. This was partitioned thrice against ethyl acetate to produce three fractions: ethyl acetate contents, the precipitate formed at the interface, and water contents. Each of these fractions were subjected to rotary evaporation and lyophilization to yield powdered extracts.

Examination of strawberry extract efficacy in vivo: For dextran efficacy studies, fasted mice were orally gavaged with 600 mg/kg STRB PPh solutions or 40 mg/kg pelargonidin solutions, then gavaged three hours later with 600 mg/kg FITC-DX4. Three hours after the dextran gavage, blood was collected and centrifuged. The serum was removed and examined for FITC concentration by reading for fluorescence on the plate reader and comparing to a unique calibration curve for each experiment. For larger macromolecule studies, 40 kDa dextran (FITC-DX40) was substituted at the same 600 mg/kg concentration.

For insulin delivery studies, mice were orally gavaged with PBS (for control) or strawberry treatments (600 mg/kg STRB PPh or 40 mg/kg pelargonidin). One hour later, their initial blood sugar was measured and the animals were placed under anesthesia. Their intestines were surgically exposed, and insulin was injected at the predetermined dose (1 unit per kg body weight unless otherwise specified) into the duodenum of the small intestine, avoiding digestion in the stomach. The mice were closed and secured with tissue adhesive, then kept under anesthesia as their blood sugar levels were monitored each hour for five hours. For comparison to the current standard of insulin delivery, subcutaneous injections were given at 1 U/kg to additional mice, into the scruff on their necks. To determine areas above the curve for each mouse, trapezoidal integration was used to sum the area between known points on the blood glucose curve and the starting blood glucose value for the individual animal.

TABLE 1
Parameters for chromatography runs.
In each case, solvent A is 0.1% TFA in water and solvent B is acetonitrile
MPLC Run α
Column: 26 × 460 mm
Starting mass: 1 g Pph

| Time (min) | % A | % B |
|---|---|---|
| 0.00 | 90 | 10 |
| 4.87 | 90 | 10 |
| 7.57 | 80 | 20 |
| 11.25 | 80 | 20 |
| 13.53 | 70 | 30 |
| 17.50 | 70 | 30 |
| 28.40 | 60 | 40 |
| 39.12 | 60 | 40 |
| 46.93 | 0 | 100 |
| 51.00 | 0 | 100 |

MPLC Runs β, γ, δ, ε
Column: 15 × 920 mm
Starting mass:
β: 0.06 g
γ: 0.04 g
δ: 0.07 g
ε: 0.12 g

| Time (min) | % A | % B |
|---|---|---|
| 0.00 | 90 | 10 |
| 6.05 | 90 | 10 |
| 14.93 | 85 | 20 |
| 20.98 | 80 | 20 |
| 28.65 | 0 | 100 |
| 32.31 | 0 | 100 |

UPLC
Acquity UPLC Column
Starting material:
10 μL MPLC fraction

| Time (min) | % A | % B |
|---|---|---|
| 0.00 | 90 | 10 |
| 1.00 | 85 | 15 |
| 3.50 | 80 | 20 |
| 6.00 | 0 | 100 |
| 7.00 | 0 | 100 |

Chromatography: Medium pressure liquid chromatography (MPLC) was performed using a Buchi Sepacore® system. Glass columns were hand-packed with reverse-phase (C18) silica gel and each run utilized a gradient from 10% acetonitrile to 100% acetonitrile in 0.1% aqueous trifluoroacetic acid (TFA). Run a was implemented for a coarse separation of the strawberry polyphenol extract (Table 1). Eluent absorption at 280 nm was monitored to track phenol group migration (Escarpa, A.; González, M. C. High-Performance Liquid Chromatography with Diode-Array Detection for the Determination of Phenolic Compounds in Peel and Pulp from Different Apple Varieties. J. Chromatogr. A 1998, 823 (1-2), 331-337. doi.org/10.1016/S0021-9673(98)00294-5). Fractions were collected, concentrated via rotary evaporation, and re-applied to a longer, narrower column for runs β, γ, δ, and ε. Fractions were collected and re-concentrated for testing in cell culture.

Each MPLC fraction was analyzed by ultra performance liquid chromatography (UPLC) using a Waters Acquity UPLC® system and Acquity UPLC C18 Column. Each run began with a 10 μl injection of concentrated MPLC eluent. The gradient began at 10% acetonitrile in 0.1% aqueous TFA, then proceeded to 100% acetonitrile (Table 1). Eluent was monitored by a photodiode array (PDA) detector, allowing each sample to be recorded for both the 280 nm absorbance trace over time and absorbance spectra of peaks.

Statistics: All data presented as arithmetic mean of the given “n” number of biological replicates (individual animals or number of in vitro cell culture wells), and error bars display the standard error of the mean. For statistical significance, two-tailed Student's t-tests were used to calculate p values.

The methods described herein exploit the ability of certain phytochemicals to safely and reversibly increase intestinal permeability. Extracts derived from over fifty fruit, vegetable, herb, and fungus samples are well-tolerated by cells of the Caco-2 human intestinal epithelial line. Furthermore, transepithelial electrical resistance (TEER), calcein permeability, and dextran permeability assays have shown that the permeability of the Caco-2 monolayer can increase, remain static, or decrease over time when exposed to treatment by different foods. TEER inversely correlates to permeability, so a reduction in TEER values implies an increase in paracellular permeability across the monolayers. Iterative separations and fraction activity screening has traced changes in barrier function to specific families of natural products. These changes to intestinal permeability were reproducible and translated successfully into more complex, animal-based models.

### Results

To screen a large number of enhancer candidates simultaneously, crude extracts of over fifty fruit and vegetable samples (Table 2) were prepared. The resulting powders were dissolved at 15 mg/ml in culture media immediately before testing with cell-based assays.

TABLE 2
Foods Prepared and Screened
as Crude Extracts

| | | |
|---|---|---|
| Aloe, | Grape, | Pomegranate |
| Culinary | Seedless Red | |
| Apple | Grapefruit | Pomelo Flesh |
| (Whole) | Flesh | |
| Apple | Grapefruit | Pomelo Rind |
| Flesh | Rind | |
| Apple | Huckleberry, | Potato, Baking |
| Peel | Garden | |
| Banana | Lemon Flesh | Potato, Blue |
| Blackberry | Lemon Rind | Potato, Red |
| Blueberry | Lime Flesh | Quince |
| Brussel | Lime Rind | Raspberry |
| Sprouts | | |
| Cabbage, | Mango | Starfruit |
| Red | | |
| Cactus | Mushrooms, | Strawberry |
| Pear | White | |
| Cantaloupe | Onion, Red | Sweet Potato |
| Carrot | Orange Flesh | Sweet Potato, |
| | | Purple |
| Carrot, | Orange Rind | Tomato, Black |
| Purple | | |
| Chayote | Passionfruit | Tomato, Red/Pink |
| Cranbeny | Pear (Whole) | Tomato, Yellow |
| Cucumber | Pear Peel | Watermelon |
| Garlic | Pepino | Wonderberry |
| Ginger | Pepper, Green | |

Before evaluation for permeation enhancement efficacy, extracts were tested for toxicity on Caco-2 cells using the PrestoBlue® viability assay. The hypothesis that edible foods would yield nontoxic extracts for intestinal cell systems was generally supported (FIG. 2); of over fifty extracts tested, only eleven showed statistically significant reductions in viability. Of the apparently toxic extracts, most were subject to extenuating circumstances, such as unintended homogenization of seeds into the extract.

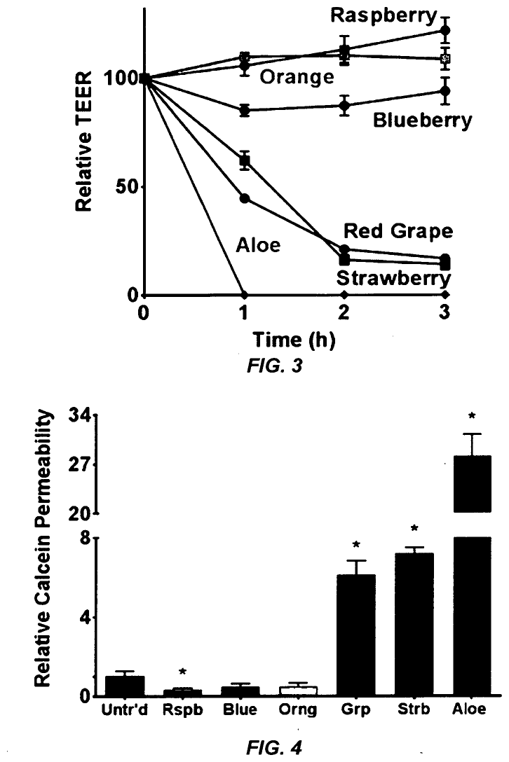

Permeation enhancement efficacy of the extracts was examined by transepithelial electrical resistance (TEER) on Caco-2 intestinal epithelial cell monolayers. Since TEER values represent the resistance of the cell monolayers to paracellular ion passage, a reduction in TEER corresponds to increased paracellular permeability of the barrier. From the extracts tested, a wide variety of resulting TEER responses were observed. Among those samples that were well-tolerated by the cells (FIG. 2), most samples did not substantially affect the TEER, while some caused significant increases or decreases. Raspberry, blueberry, and orange yielded high TEER values through the entire treatment, corresponding to a strengthening of tight junction integrity. By contrast, culinary aloe, red grapes, and strawberries substantially reduced TEER and increased permeability of the cell layers (FIG. 3). However, among these three most active permeation enhancers, only the effects of red grapes and strawberries were reversible after a 24-hour recovery period; the Caco-2 monolayers treated with aloe did not recover barrier function after treatment removal, indicating permanent changes or damage to the cells.

To confirm the tight junction modulations implied by TEER, apparent permeability of calcein through the Caco-2 monolayers was also assessed (FIG. 4). Due to its strong negative charge at physiological pH (=7.4), calcein must transport paracellularly, in the same fashion as macromolecule drugs. As in TEER experiments, raspberry, blueberry, and orange strengthened the barrier function of Caco-2 tight junctions, while aloe, strawberry, and red grape were identified as potential permeability enhancers.

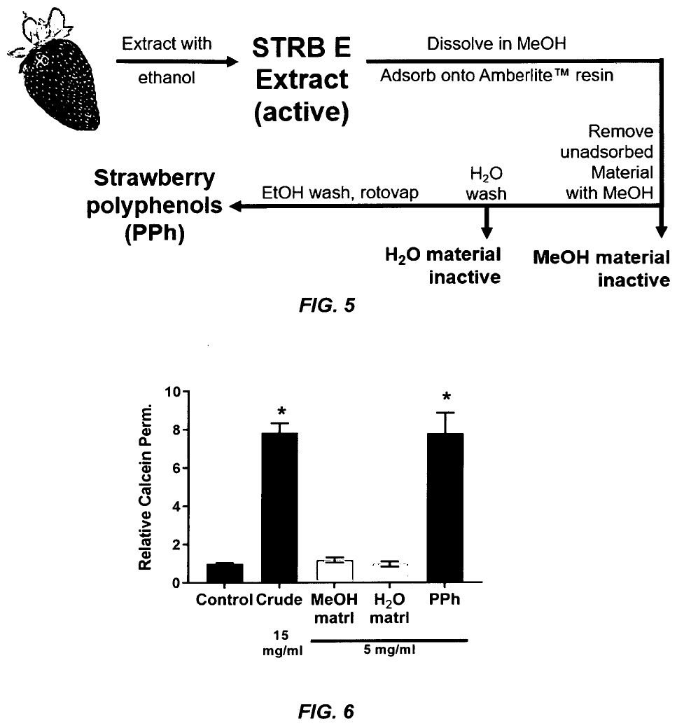

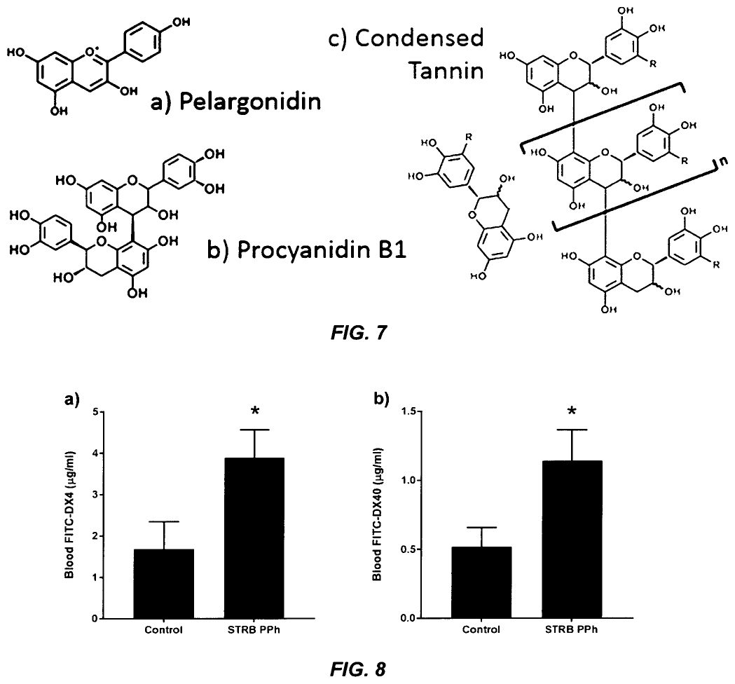

As a result of strawberry's supreme combination of reversibility and efficacy, it was chosen for separation techniques, with an end goal of determining the specific active components that induce epithelial permeation enhancement. Crude strawberry extracts were extracted with 80% aqueous ethanol and subjected to adsorption-based separation on Amberlite XAD 7 HP acrylate resin (FIG. 5), resulting in three fractions. While the unabsorbed (MeOH material) and extremely hydrophilic (H₂O material) products showed little activity, the fraction that adsorbed strongly to the resin caused significant increases in calcein permeability through treated Caco-2 monolayers (FIG. 6). This indicates that AMBERLITE™ separation is an effective step in dividing active from inactive material in the strawberry extracts. Based on the chemistry of AMBERLITE™ resins and the results of electrospray ionization mass spectrometry (ESI-MS) on these active fractions, they are likely composed of polyphenolics (FIG. 7).

To confirm that permeation enhancement in Caco-2 correlates to improved macromolecular bioavailability through intestinal tissues, mice were orally dosed with strawberry polyphenols, followed by 4 kDa, FITC-labelled dextran (FITC-DX4). FITC-DX4 is a non-digestible, fluorescent macromolecule commonly used to probe the permeability of the intestinal epithelium in animal models. After three hours, the serum fluorescence was measured to determine the blood FITC-DX4 concentration. As expected, the strawberry polyphenols (STRB PPh) increased FITC-DX4 absorption across the intestinal barrier by more than 100% (FIG. 8a). The same double in uptake efficiency was seen when 40 kDa FITC-dextran (FITC-DX40) was delivered orally (FIG. 8b), indicating that the strawberry polyphenols can be used to deliver a wide size range of macromolecule drugs.

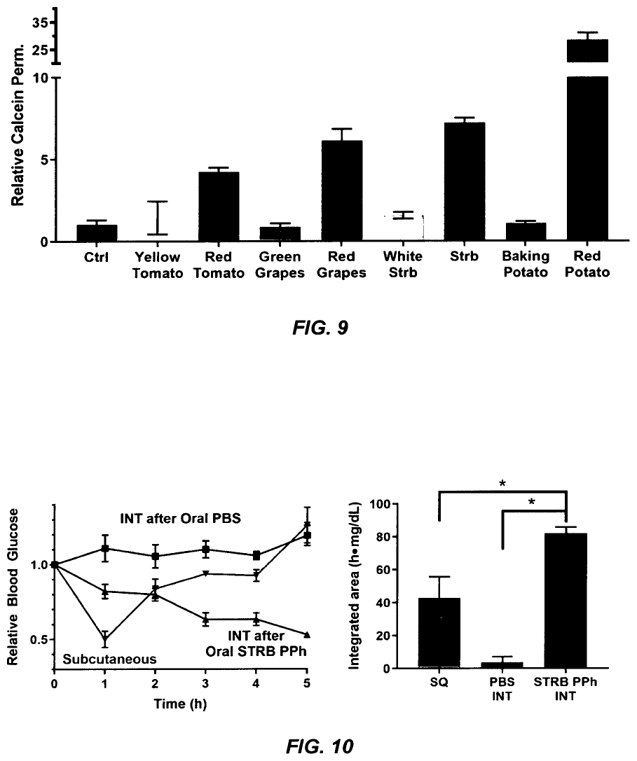

Among the screening results from the crude extract library, an intriguing trend emerged: different colored varieties of the same fruits and vegetables sometimes yielded drastically different permeability responses. This was not true for all sets of varieties tested; in the cases of apples (red and green), cherries (red and white), and peaches (standard and white), none of the extracts were effective permeation enhancers. However, for tomatoes, grapes, strawberries, and potatoes, there was a visible pattern: the red varieties of those foods were all effective permeation enhancers, while the white, green, or yellow varieties did not substantially affect monolayer permeability (FIG. 9). Interestingly, each of those crops are known to produce a family of molecules, called polyphenols, which often contribute to plant coloration. Based on this connection, we decided to examine this family as the next step in our iterative isolation process. Because strawberry was the most efficacious yet reversible permeabilizing extract (monolayers treated with red potato did not recover their barrier function), we proceeded to examine strawberry polyphenols as our target molecules.

As a proof-of-concept that improved dextran absorption would correlate with improved oral protein delivery, we examined transport of functional insulin across the mouse epithelium. We chose insulin for these studies because it is a modestly-sized (5.8 kDa) and relatively inexpensive protein that is not orally bioavailable in normal animals. Furthermore, its bioactivity is easily assessed by monitoring the depression of blood glucose levels that results from increased insulin concentration in the bloodstream. Mice received an oral dose of strawberry polyphenols, followed by an injection of 1 U/kg dose of insulin directly into the small intestine, circumventing possible digestion in the stomach. Blood glucose levels were monitored each hour and normalized to each mouse's blood sugar before the procedure. Mice that received insulin and strawberry polyphenols experienced a substantial reduction in blood glucose compared to mice that received insulin after just a saline gavage (FIG. 10, left). Further, the STRB PPh and intestinal insulin combination sustained hypoglycemia several hours longer than the same 1 U/kg dose of subcutaneous insulin, the current gold standard of administration.

To compare the total insulin bioactivity between these administration methods, we integrated the areas between each mouse's glucose curve and its starting blood sugar value. The areas above the curve (AACs) show that pharmacodynamic activity of intestinal insulin in polyphenol-treated mice is approximately double that of subcutaneous insulin (FIG. 10, right), yielding a relative bioactivity value of 191%. Importantly, the sustained activity of the polyphenol-assisted intestinal insulin indicates that this administration route may be advantageous for drugs that require extended release profiles. Additionally, this successful protein absorption across the intestinal epithelium confirmed that strawberry polyphenols are efficacious enhancers and should be further pursued and refined for this purpose.

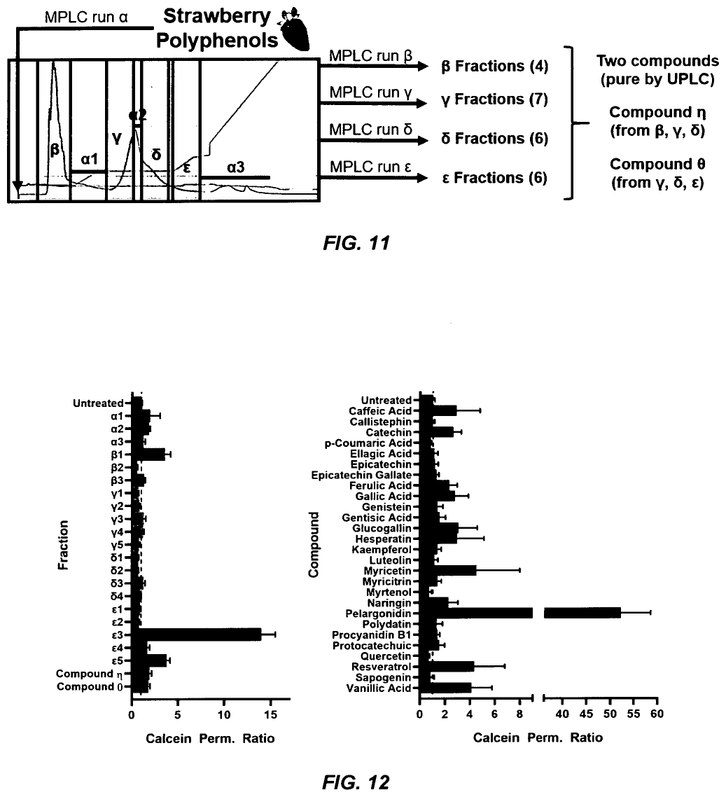

To continue the iterative separation-activity cycle of compound isolation, we turned to chromatography for better resolution of the strawberry polyphenol extract into discrete fractions. Specifically, we employed reverse-phase medium pressure liquid chromatography (MPLC) using a trifluoroacetic acid (TFA)-doped water and acetonitrile mobile phase. Each MPLC run was assigned a Greek letter identifier, moving consecutively though the alphabet. The first MPLC run (α) yielded fractions that were divided into seven groups, based on fraction color and ultra-performance liquid chromatography (UPLC) traces of the eluents (FIG. 11). Four of these produced enough material to support a second tier of MPLC runs, yielding fractions for the β, γ, δ, and ε groups, respectively. Interestingly, UPLC traces identified two pure compounds that each appeared in multiple Greek letter groups. These were pooled together to yield the samples denoted as Compound r and Compound 6.

Next, we took each of the 22 fractions and compounds from MPLC and screened them for bioactivity on Caco-2 monolayers. Most fractions were screened at a concentration of 1 mg/mL, though some (β2, γ1, δ2, ε1, and ε3) did not yield enough material and were tested at lower concentrations (entire fraction). By calcein permeability, the vast majority of the samples did not substantially affect epithelial permeability (FIG. 12, left). However, one fraction, ε3, was an exceptional permeation enhancer. Interestingly, ε3's late elution from the columns identifies is as one of the more hydrophobic members of the polyphenol family. It was also deep red in color and a very small fraction, yielding less than three milligrams from one gram of polyphenol starting material (likewise, pelargonidin makes up approximately 0.001% of the fresh weight of the strawberries. While the small mass promisingly indicated the fraction's high potency, it also complicated efforts to properly discern its molecular identity.

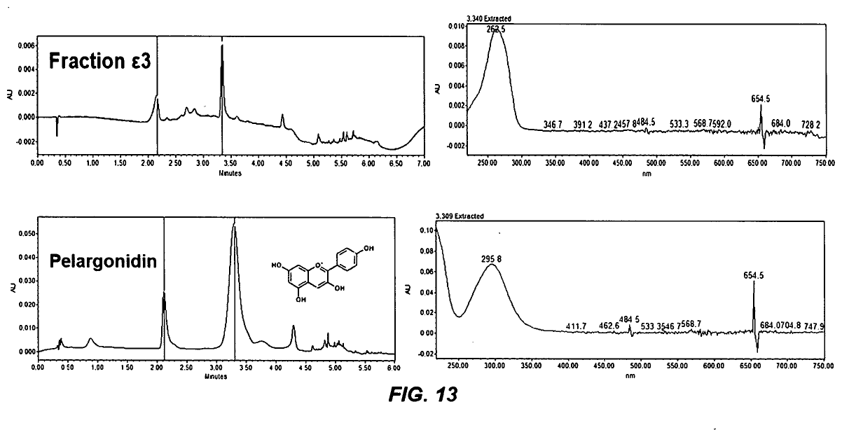

To expedite our identification of the permeation enhancing compound in fraction ε3, we assembled a library of known phenolic compounds from strawberry. As with the fractions, we screened these for their bioactivity on Caco-2 cells at 1 mg/mL concentration (FIG. 12, right). By calcein permeability, only one of these compounds was an effective permeation enhancer: pelargonidin. Pelargonidin and its glucoside, callistephin, are primarily responsible for the red coloration in strawberries (Kosińska-Cagnazzo, A.; Diering, S.; Prim, D.; Andlauer, W. Identification of Bioaccessible and Uptaken Phenolic Compounds from Strawberry Fruits in in Vitro Digestion/Caco-2 Absorption Model. Food Chem. 2015, 170, 288-294. doi.org/10.1016/j.foodchem.2014.08.070 and Seeram, N. P.; Lee, R.; Scheuller, H. S.; Heber, D. Identification of Phenolic Compounds in Strawberries by Liquid Chromatography Electrospray Ionization Mass Spectroscopy. Food Chem. 2006, 97 (1), 1-11. doi.org/10.1016/j.foodchem.2005.02.047). The vast majority of the pigment is present in the glycosylated form, due to its improved solubility for storage in aqueous vacuoles. However, our screening showed that callistephin was not an effective permeation enhancer (FIG. 12, right), and that only the non-glycosylated pelargonidin improved epithelial permeability. This is consistent with the observations that the effective fraction, ε3, yielded a small mass of deep red, fairly hydrophobic material. To further support the similarity between ε3 and pelargonidin, we ran a sample of each on UPLC and compared the trace at 280 nm, as well as the absorbance spectra of the peaks (FIG. 13). While it should be noted that the pelargonidin standard was run with almost an order of magnitude more material, the UPLC readouts were nonetheless consistent. Both samples displayed characteristic peaks in 280 nm absorbance at approximately 2.1 and 3.3 minutes into the run, as well as similar light absorption spectra. While this evidence is sufficient for us to conclude that pelargonidin is the primary permeation enhancer in strawberries, ongoing work aims to further probe its bioactivity and irrefutably confirm the identity of the ε3 fraction via NMR analysis.

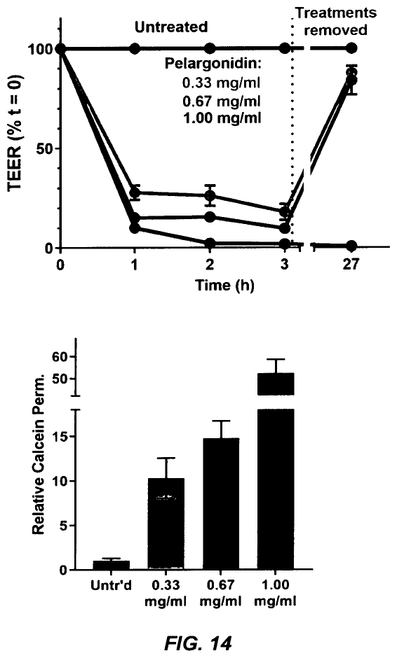

In addition to strawberries, pelargonidin can be found in each of the previously-discussed red foods: tomatoes, red grapes, and red potatoes. With this knowledge and our chromatography-based evidence, we set out to demonstrate and characterize pelargonidin as a reversible, food-derived permeation enhancer. First, we examined dose dependent permeability response in Caco-2 monolayers. Reductions in TEER (FIG. 14, left) and improvements in calcein permeability (FIG. 14, right) both increased with higher concentrations of pelargonidin treatment. It should be noted that the highest concentration, 1 mg/ml pelargonidin, opened the tight junctions beyond their ability to recover barrier function, underscoring the potency of this molecule at low concentrations. However, both lower concentrations of pelargonidin, 0.33 mg/ml and 0.67 mg/ml, induced substantial yet reversible permeabilization of the monolayers.

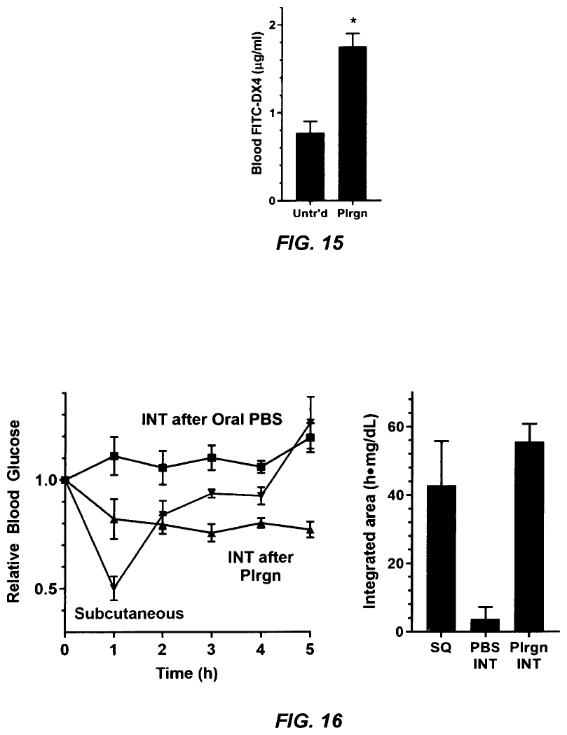

We then asked whether the pelargonidin-induced improvements in Caco-2 monolayer permeability would translate into successful oral administration of proteins and other macromolecules. To test this, we administered pelargonidin (40 mg/kg), then FITC-DX4 orally to mice, then examined the content of the fluorescent dextran in their blood. As expected, pelargonidin treatment doubled FITC-DX4 absorption across the intestinal barrier (FIG. 15) when compared to a phosphate-buffered saline (PBS) gavage. Based on this improved oral bioavailability of FITC-DX4, we can conclude that pelargonidin is an effective intestinal permeation enhancer that holds promise for future development and clinical translation.

To ensure that pelargonidin would enable oral delivery of insulin, mice received an oral dose of pelargonidin (40 mg/kg), followed by an injection of 1 U/kg dose of insulin directly into the small intestine, circumventing possible digestion in the stomach. Blood glucose levels were monitored each hour and normalized to each mouse's blood sugar before the procedure. Mice that received insulin and pelargonidin experienced a substantial reduction in blood glucose compared to mice that received insulin after just a saline gavage (FIG. 16, left). Further, the pelargonidin and intestinal insulin combination sustained hypoglycemia several hours longer than the same 1 U/kg dose of subcutaneous insulin. The areas above the curve (AACs) show that pharmacodynamic activity of intestinal insulin in polyphenol-treated mice is approximately 1.3 times that of subcutaneous insulin (FIG. 16, right), yielding a relative bioactivity value of 130%.

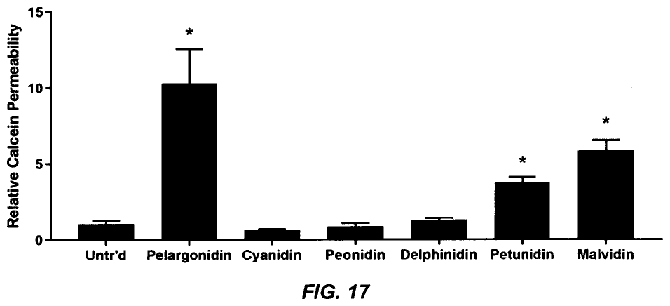

There are several members of the anthocyanidin family that, like pelargonidin, occur in food and plant tissues (Ananga, A.; Georgiev, V.; Ochieng, J.; Phills, B.; Tsolova, V. Production of Anthocyanins in Grape Cell Cultures: A Potential Source of Raw Material for Pharmaceutical, Food, and Cosmetic Industries. The Mediterranean Genetic Code: Grapevine and Olive, edited by Danijela Pojuha and Barbara Sladonja, Intech, 2013, pp. 247-286. doi.org/10.5772/54592). Application of these molecules, which included cyanidin, peonidin, delphinidin, petunidin, and malvidin, to Caco-2 monolayers indicated that at least two additional members of this family are effective permeation enhancers (FIG. 17). Specifically, in addition to pelargonidin, petunidin and malvidin increase the permeability of calcein across Caco-2 intestinal monolayers with statistical significance. Pelargonidin, petunidin, and malvidin will be produced in bulk for further testing in mice, which will examine oral delivery of dextrans, insulin, other commercially significant protein drugs such as antibodies, and macromolecular carbohydrates such as heparin and inulin.

Additionally, while the anthocyanins (glycosylated anthocyanidins such as callistephin) did not effectively permeabilize Caco-2 monolayers, their glyosidic bond is known to cleave in acidic conditions with heating (Corona, G.; Tang, F.; Vauzour, D.; Rodriguez-Mateos, A.; Spencer, J. P. E. Assessment of the anthocyanidin content of common fruits and development of a test diet rich in a range of anthocyanins. J Berry Res. 2011, (1), 209-216. doi.org/10.3233/BR-2011-022). As this combination of physical and chemical conditions closely mimics stomach conditions, it is possible that anthocyanins will be cleaved to yield active anthocyanidins when orally administered in an animal model. This will be examined as a potential strategy for delivering the anthocyanidin permeation enhancers.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

1. A dosage form comprising:
a therapeutic agent ranging in size from 10 Da to 150 kDa; and
a polyphenol-containing composition, comprising a polyphenol or a mixture of plant polyphenols able to increase permeability of the intestinal epithelium in a patient, and in an amount effective to increase permeability of intestinal epithelium of a patient.

2. The dosage form of claim 1, wherein the polyphenol-containing composition is prepared from a red-pigmented plant material, a pelargonidin-containing plant material, a petunidin-containing plant material, or a malvidin-containing plant material, or from strawberry fruit, red tomato, red potato, red grape skin, or a geranium flower.

3. The dosage form of claim 1, wherein the polyphenol-containing composition is liquidized or crushed fruit, fruit skin, or flower, or dried fruit, fruit skin, or flower.

4. (canceled)

5. The dosage form of claim 1, wherein the polyphenol-containing composition is a polyphenol extract of plant material, or an anthocyanidin-enriched or anthocyanin-enriched fraction prepared from plant material.

6. (canceled)

7. The dosage form of claim 1, wherein the polyphenol-containing composition comprises:
pelargonidin or a glycoside thereof;
petunidin or a glycoside thereof;
malvidin or a glycoside thereof;
isolated pelargonidin;
a pelargonidin enriched fraction obtained from a natural plant material;
a mixture of isolated or enriched polyphenols comprising pelargonidin;
isolated petunidin;
a petunidin-enriched fraction obtained from a natural plant material;
a mixture of isolated or enriched polyphenols comprising petunidin;
isolated malvidin;
a malvidin-enriched fraction obtained from a natural plant material;
a mixture of isolated or enriched polyphenols comprising malvidin; or
any combination thereof, in an amount effective to increase permeability of intestinal epithelium of a patient.

8. The dosage form of claim 1, wherein the polyphenol-containing composition is a fraction prepared from a fruit, fruit skin, or flower that is retained on a hydrophobic resin in water and is eluted from the resin in ethanol, and wherein the hydrophobic resin is optionally a macroreticular aliphatic crosslinked polymer composition.

9. The dosage form of claim 1, wherein the therapeutic agent is insulin, which is optionally provided in an amount ranging from 5 IU insulin to 200 IU insulin per unit dose.

10. The dosage form of claim 1, wherein the therapeutic agent is a protein.

11. The dosage form of claim 1, wherein the therapeutic agent is one or more of abaloparatide, adrenocorticotropic hormone, afamelanotide, albiglutide, ambamustine, atosiban, aviptadil, buserelin, carbetocin, carfilzomib, carperitide, cetrorelix, cholecystokinin, calcitonin (salmon or human), carperitide, corticotropin, cyclosporine, degarelix, desmopressin, dulaglutide, elcatonin, eledoisin, enalapril, enfuvirtide, etelcalcetide, exenatide, felypressin, ganirelix, glatiramer, glucagon, glucagon-like peptide 2, glucose-dependent insulinotropic peptide, gonadorelin, goserelin, heparin, histrelin, human growth hormone, icatibant, lanreotide, leuprorelin (leuprolide), linaclotide, liraglutide, lisinopril, lixisenatide, lucinactant, lutetium, lypressin, mifamurtide, nafarelin, nesiritide, octreotide, ornipressin, oxytocin, pasireotide, plecanatide, pramlintide, romiplostim, romurtide, somatostatin, taltirelin, teduglutide, teriparatide, terlipressin, tetracosactide, thymopentin, triptorelin, vasopression, virus capsid proteins and other antigens, voclosporin, or ziconotide. Specific active ingredients in the binding agents class deliverable by the methods, dosage forms, or compositions described herein include, without limitation: abciximab, adalimumab, alefacept, alemtuzumab, alirocumab, altumomab pentetate, arcitumomab, atezolizumab, avelumab, basiliximab, bectumomab, belimumab, benralizumab, bermekimab, besilesomab, bevacizumab, bezlotoxumab, biciromab, blinatumomab, blontuvetmab, brentuximab vedotin, brodalumab, burosumab, canakinumab, caplacizumab, capromab pendetide, removab, cemiplimab, certolizumab pegol, cetuximab, clivatuzumab tetraxetan, cosfroviximab, daclizumab, daratumumab, denosumab, dinutuximab, dupilumab, durvalumab, eculizumab, edrecolomab, efalizumab, elotuzumab, emapalumab, emicizumab, erunumab, ertumaxomab, etanercept, etracizumab, evolocumab, famolesomab, fantolizumab, fremanezumab, galcanezumab, gemtuzumab ozogamicin, girentuximab, golimumab, guselkumab, ibalizumab, ibritumomab tiuxetan, idarucizamab, igovomab, imciromab, ipilimumab, itolizumab, ixekizumab, labetuzumab, lanadelumab, loviketmab, mepolizumab, mogamulizumab, motavizumab, muromonab-CD3, naptumomab estefenatox, natalizumab, necitumumab, nimotuzumab, nivolumab, nofetumomab, oblitoxaximab, obinutuzumab, ocrelizumab, ofatumumab, olaratumab, omalizumab, oportuzumab, oregovomab, palivizumab, panitumumab, pembrolizumab, pemtumomab, pertuzumab, racotumomab, ramucirumab, ranibizumab, ravulizumab, reslizumab, risankizumab, rituximab, romosozumab, rovelizumab, ruplizumab, sarilumab, secukinumab, siltuximab, sulesomab, tamtuvetmab, tefibazumab, tildrakizumab, toclizumab, tositumomab, trastuzumab, ustekinumab, vedolizumab, visilizumab, votumumab, zalutumumab, or zanolimumab.

12. The dosage form of claim 1, wherein the therapeutic agent is a binding reagent, optionally chosen from an F(ab) fragment, an F(ab′)₂ fragment, an F(ab′) fragment, an scFv (single-chain variable fragment), a di-scFv (dimeric single-chain variable fragment), a bi-specific T-cell engager (BiTE), a single-domain antibody (sdAb), or an antibody binding domain, or a carbohydrate such as a polysaccharide or a glycosaminoglycan, such as heparin.

13. (canceled)

14. The dosage form of claim 1 in the form of an oral, enteric-coated dosage form, or a gastrointestinal dosage form.

15. A method of delivering an active agent to a patient, comprising:
enterically delivering to a patient an amount of a polyphenol-containing composition comprising a polyphenol or a mixture of plant polyphenols able to increase permeability of the intestinal epithelium in a patient, in an amount effective to increase permeability of intestinal epithelium of a patient; and
delivering a therapeutic agent ranging in size from 10 Da to 150 kDa to the intestine at the same time as the delivery of the polyphenol-containing composition to the patient, within 6, 5, 4, 3, 2, or 1 hour, or 30 minutes of delivery of the polyphenol-containing composition to the patient, or during a time period where the permeability of the intestinal epithelium of the patient is increased due to the presence of the polyphenol-containing composition.

16. The method of claim 15, wherein the polyphenol-containing composition is prepared in a dosage form comprising:
a therapeutic agent ranging in size from 1,000 Da to 150 kDa; and
a polyphenol-containing composition, comprising a polyphenol or a mixture of plant polyphenols able to increase permeability of the intestinal epithelium in a patient, and in an amount effective to increase permeability of intestinal epithelium of a patient.

17. (canceled)

18. (canceled)

19. (canceled)

20. (canceled)

21. The method of claim 15, wherein the polyphenol-containing composition comprises:
pelargonidin or a glycoside thereof;
petunidin or a glycoside thereof; or
malvidin or a glycoside thereof.

22. (canceled)

23. The method of claim 15, wherein the therapeutic agent is insulin, which is optionally provided in an amount ranging from 5 IU insulin to 200 IU insulin per unit dose.

24. The method of claim 15, wherein the therapeutic agent is a protein.

25. (canceled)

26. The method of claim 15, wherein the therapeutic agent is a binding reagent, optionally chosen from an F(ab) fragment, an F(ab′)₂ fragment, an F(ab′) fragment, an scFv (single-chain variable fragment), a di-scFv (dimeric single-chain variable fragment), a bi-specific T-cell engager (BiTE), a single-domain antibody (sdAb), or an antibody binding domain; a carbohydrate; or a glycosaminoglycan.

27. (canceled)

28. The method of claim 15, wherein the polyphenol-containing composition and the therapeutic agent are delivered orally or gastrointestinally.

29. (canceled)

30. A method of reversibly increasing intestinal epithelium permeability in a patient in need thereof, comprising, delivering to an intestine of a patient an amount of a polyphenol-containing composition comprising a polyphenol or a mixture of plant polyphenols able to increase permeability of the intestinal epithelium in a patient, in an amount effective to increase permeability of intestinal epithelium of a patient, wherein the polyphenol-containing composition comprises:
isolated pelargonidin;
a pelargonidin-enriched fraction obtained from a natural plant material;
a mixture of isolated or enriched polyphenols comprising pelargonidin;
isolated petunidin;
a petunidin-enriched fraction obtained from a natural plant material;
a mixture of isolated or enriched polyphenols comprising petunidin;
isolated malvidin;
a malvidin-enriched fraction obtained from a natural plant material;
a mixture of isolated or enriched polyphenols comprising malvidin; or
any combination thereof.

31. A method of treating hyperglycemia or diabetes in a patient, comprising administering an amount of the dosage form of claim 9 to the patient effective to treat hyperglycemia or diabetes in the patient.
